(19) United States
(12) Patent Application Publication
(10) Pub. No.: US 2023/0131376 A1
(43) Pub. Date: Apr. 27, 2023

(54) ADJUVANT-ANTIBIOTIC COMBINATION AGAINST GRAM-NEGATIVE BACTERIA

(71) Applicants: Christian MELANDER, South Bend, IN (US); Bradley M. MINROVIC, South Bend, IN (US); University of Notre Dame du Lac, South Bend, IN (US)

(72) Inventors: Christian MELANDER, South Bend, IN (US); Bradley M. MINROVIC, South Bend, IN (US)

(21) Appl. No.: 17/908,506

(22) PCT Filed: Apr. 16, 2020
(86) PCT No.: PCT/US2020/028484

§ 371 Date: Aug. 31, 2022

(51) Int. Cl. A61K 38/12; A61K 31/4045

(52) U.S. Cl. CPC A61K 38/12; A61K 31/4045

(57) ABSTRACT

Tryptamine ureas and derivatives function as adjuvants to sensitize gram-negative bacteria to the effects of polymyxin antibiotics (e.g., colistin). Combination therapy of polymyxin antibiotics and the adjuvants has utility in the treatment of gram-negative bacterial infection, including treatment of drug-resistant strains.

### STATEMENT OF GOVERNMENT INTEREST

This invention was made with government support under grant numbers GM055769 and R01 AI136904, awarded by the-National Institutes of Health. The government has certain rights in the invention.

### TECHNICAL FIELD

The present invention relates to tryptamine ureas and derivatives having adjuvant activity to sensitize gram-negative bacteria to the effects of polymyxin antibiotics.

### BACKGROUND

Antibiotics have significantly contributed to the quality of life since the discovery of penicillin in the 1930s. Despite the subsequent discovery of numerous antibiotic classes spanning multiple modes of action, bacteria have acquired resistance to every antibiotic in the clinician's arsenal. Antibiotic resistance will likely continue to become more common, as antibiotic usage rose 65% from 2000 to 2016, and history has shown us that resistance is more likely to be acquired when antibiotic consumption is increased. Currently, the Centers for Disease Control and Prevention estimates approximately two million people present with an antibiotic resistant infection annually in the U.S., and the death toll from these infections is estimated to be at least 23,000. A majority of these fatal infections are caused by the ESKAPE pathogens: the Gram-positive bacteria Enterococcus faecium and Staphylococcus aureus, and the Gram-negative bacteria Klebsiella pneumoniae, Acinetobacter baumannii, Pseudomonas aeruginosa, and Enterobacter spp.

The arduous nature of developing effective therapies to treat multidrug-resistant (MDR) Gram-negative bacteria has been well documented⁴ and is hampered by the presence of both intrinsic and acquired resistance mechanisms that can render bacteria resistant to nearly, if not all, antibiotics on the market. Indeed, in 2017, a woman in the United States succumbed to septic shock that stemmed from an infection caused by a strain of K. pneumoniae that was resistant to 26 antibiotics, including all aminoglycosides and polymyxins tested.

The escalation of carbapenem resistance in Gram-negative bacteria has resulted in the increased usage of the “last resort” antibiotic colistin, a macrocylic cationic polypeptide with a bactericidal mechanism that involves direct physical disruption of the Gram-negative outer membrane. Colistin is, however, nephrotoxic with ca. 25% of patients who are administered a full regimen experiencing some degree of nephrotoxicity (this does not include patients whose regimen was halted prematurely due to toxicity). For this reason, physicians have traditionally avoided the use of colistin; however, the rise in resistance to other more well-tolerated antibiotic classes has forced colistin usage as of late. Prior to 2016, all known clinical resistance to colistin was mediated by chromosomally-encoded genes. This situation changed dramatically with a report in 2016 of a patient presenting with a colistin resistant Escherichia colt infection, in which the gene (mcr-1) encoding the colistin resistance machinery was located on a plasmid, raising the possibility that colistin resistance could become rapidly widespread through lateral gene transfer. Indeed, mcr-1 and its variant genes (mcr-2-8) have now been reported globally. The mcr-1 gene encodes a phosphoethanolamine transferase that covalently modifies lipid A with phosphoethanolamine, which serves to decrease the overall net negative charge of the membrane and greatly impacts the ability of colistin to disrupt the bacterial membrane due to decreased electrostatic interactions.

As the antibiotic pipeline for the treatment of MDR Gram-negative infections is sparse, the development of novel approaches to combat these bacteria are warranted. One such method is the antibiotic-adjuvant approach, in which a conventional antibiotic is co-dosed with a non-toxic small molecule that increases the antibiotic's efficacy (Brackett et al., Tetrahedron 2016, 72 (25), 3549-3553; Harris et al., ACS Chem Biol 2014, 9 (1), 122-127). In a one study, tryptamine derivative 1a exhibited a 32-fold and a

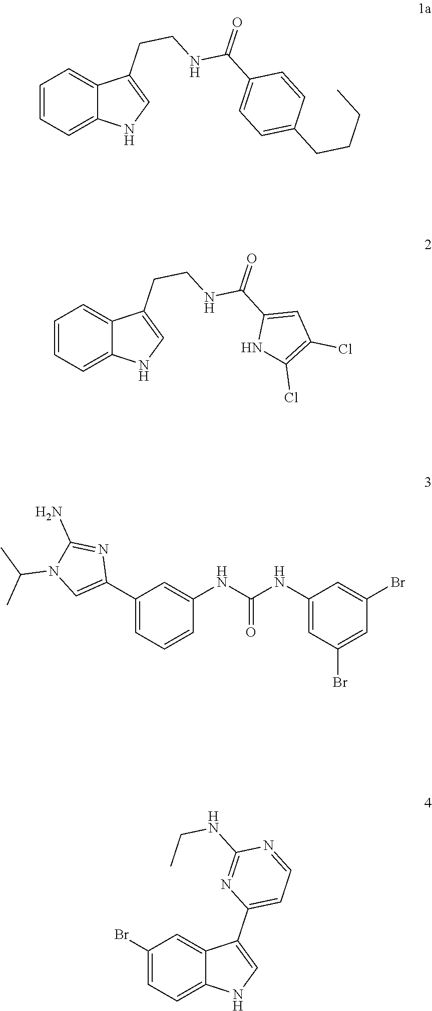 [SMILES: CCCCc1ccc(C(=O)NCCc2c[nH]c3ccccc23)cc1 ; O=C(NCCc1c[nH]c2ccccc12)c1cc(Cl)c(Cl)[nH]1 ; CC(C)n1cc(-c2cccc(NC(=O)Nc3cc(Br)cc(Br)c3)c2)nc1N ; CCNc1nccc(-c2c[nH]c3ccc(Br)cc23)n1]

two-fold reduction in the colistin minimum inhibitory concentration (MIC) against Francisella philomiragia and F. novicida respectively when tested at 50 μM (Stephens et al., Medchemcomm 2016, 7 (1), 128-131). Additional indole and 2-aminoimidazole (2-AI) derivatives (2, 3, and 4) have also demonstrated the ability to potentiate colistin activity against several MDR Gram-negative bacterial strains (Barker et al., Bioorg Med Chem 2017, 25 (20), 5749-5753; Huggins et al., ACS Med Chem Lett 2018, 9 (7), 702-707; Minrovic et al., ACS Infect Dis 2018, 4 (9), 1368-1376).

### SUMMARY

In one aspect, the invention provides a method of treating a gram-negative bacterial infection comprising administering to a subject in need thereof, a polymyxin antibiotic, or a pharmaceutically acceptable salt thereof, and a compound of formula (I) in amounts that together are effective to inhibit the gram-negative bacterial infection,

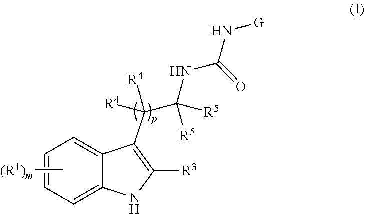

wherein
R¹ is halogen, cyano, CH₃, OCH₃, OCF₃, or OCHF₂;
G is a 6- to 12-membered aryl optionally substituted with 1-5 R² substituents;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, or —OC₃₋₆cycloalkyl;
R³ is hydrogen, C₁₋₄alkyl, C₁₋₄haloalkyl, or C₃₋₆cycloalkyl;
R⁴, at each occurrence, is independently hydrogen, C₁₋₄alkyl, C₁₋₄haloalkyl, or C₃₋₆cycloalkyl;
R⁵, at each occurrence, is independently hydrogen, C₁₋₄alkyl, C₁₋₄haloalkyl, or C₃₋₆cycloalkyl;
p is 0 or 1; and
m is 0, 1, or 2.

In another aspect, the invention provides a method of potentiating the activity of a polymyxin antibiotic against a gram-negative bacterial infection comprising administering to a subject in need thereof a compound of formula (I) in an amount effective to increase the therapeutic effect of a dose of the polymyxin antibiotic, or a pharmaceutically acceptable salt thereof, compared to the therapeutic effect of the dose of the polymyxin antibiotic, or a pharmaceutically acceptable salt thereof, in the absence of the compound of formula (I), as defined herein.

In another aspect, the invention provides a use of a pharmaceutical combination of a polymyxin antibiotic, or a pharmaceutically acceptable salt thereof, and a compound of formula (I) for the preparation of a medicament for the treatment of a gram-negative bacterial infection.

In another aspect, the invention provides a use of a pharmaceutical combination of a polymyxin antibiotic, or a pharmaceutically acceptable salt thereof, and a compound of formula (I) for the preparation of a medicament for potentiation of the activity of a polymyxin antibiotic against a gram-negative bacterial infection.

In another aspect, the invention provides a compound of formula (IV)

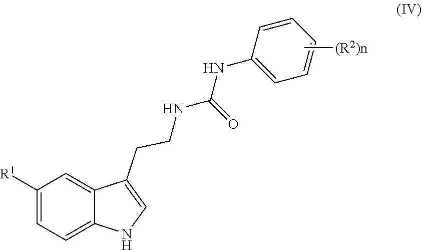

wherein
R¹ is halogen, cyano, CH₃, OCH₃, OCF₃, or OCHF₂;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, —OC₃₋₆cycloalkyl; and
n is 0, 1, 2, 3, 4, or 5;
provided that the compound of formula (IV) is not:
N-[2-(5-fluoro-1H-indol-3-yl)ethyl]-N′-(4-methoxyphenyl)-urea;
N-(2-fluorophenyl)-N′-[2-(5-methyl-1Hindol-3-yl)ethyl]-urea;
N-(3-fluorophenyl)-N′-[2-(5-methyl-1Hindol-3-yl)ethyl]-urea;
N-(4-ethoxyphenyl)-N′-[2-(6-methoxy-1Hindol-3-yl)ethyl]-urea;
N-[2-(5-chloro-1H-indol-3-yl)ethyl]-N′-(2,4-dimethoxyphenyl)-urea;
N-(3,5-dimethoxyphenyl)-N′-[2-(5-fluoro-1H-indol-3-yl)ethyl]-urea;
N-[2-(5-fluoro-1H-indol-3-yl)ethyl]-N′-(3-methylphenyl)-urea;
N-(5-chloro-2-methoxyphenyl)-N′-[2-(5-fluoro-1H-indol-3-yl)ethyl]-urea;
N-(3-bromophenyl)-N′-[2-(5-fluoro-1Hindol-3-yl)ethyl]-urea;
N-(3-chlorophenyl)-N′-[2-(5-fluoro-1Hindol-3-yl)ethyl]-urea;
N-(4-chlorophenyl)-N′-[2-(5-fluoro-1Hindol-3-yl)ethyl]-urea;
N-(3,4-dichlorophenyl)-N′-[2-(5-fluoro-1Hindol-3-yl)ethyl]-urea;
N-[2-(5-fluoro-1H-indol-3-yl)ethyl]-N′-(4-methylphenyl)-urea;
N-[2-(5-fluoro-1H-indol-3-yl)ethyl]-N′-phenyl-urea;
N-[2-(5-chloro-1H-indol-3-yl)ethyl]-N′-(4-methylphenyl)-urea;
N-[2-(5-methyl-1H-indol-3-yl)ethyl]-N′-(4-methylphenyl)-urea;
N-[2-(5-chloro-1H-indol-3-yl)ethyl]-N′-(4-fluorophenyl)-urea;
N-[2-(5-chloro-1H-indol-3-yl)ethyl]-N′-(4-nitrophenyl)-urea;
N-[2-(5-chloro-1H-indol-3-yl)ethyl]-N′-(3-chlorophenyl)-urea;
N-[2-(5-chloro-1H-indol-3-yl)ethyl]-N′-(3,4-dichlorophenyl)-urea;
N-[2-(5-chloro-1H-indol-3-yl)ethyl]-N′-(4-chlorophenyl)-urea;
N-[2-(5-chloro-1H-indol-3-yl)ethyl]-N′-phenyl-urea;
N-(4-fluorophenyl)-N′-[2-(5-methyl-1Hindol-3-yl)ethyl]-urea;
N-[2-(5-methyl-1H-indol-3-yl)ethyl]-N′-(4-nitrophenyl)-urea;
N-(3-chlorophenyl)-N′-[2-(5-methyl-1Hindol-3-yl)ethyl]-urea;
N-(3,4-dichlorophenyl)-N′-[2-(5-methyl-1Hindol-3-yl)ethyl]-urea;
N-(4-chlorophenyl)-N′-[2-(5-methyl-1Hindol-3-yl)ethyl]-urea; or
N-[2-(5-methyl-1H-indol-3-yl)ethyl]-N′-phenyl-urea.

In another aspect, the invention provides a compound of formula (III)

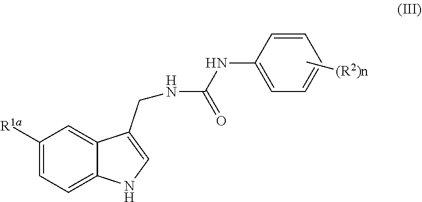

wherein
R^{1a} is hydrogen, halogen, cyano, CH₃, OCH₃, OCF₃, or OCHF₂;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, or —OC₃₋₆cycloalkyl; and
n is 0, 1, 2, 3, 4, or 5.

In another aspect, the invention provides a pharmaceutical composition comprising a compound of any of formulas (I), (II), (III), or (IV), and a pharmaceutically acceptable carrier.

### BRIEF DESCRIPTION OF THE FIGURES

FIG. 1 shows Kaplan-Meier curves of worms inoculated with 6×10⁵ colony forming units of A. baumannii 4106 and treated with tigecycline, colistin, compound 9e, or colistin and compound 9e.

FIG. 2 shows cell viability curves for compounds 8b-d, 9d-e, and 14.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} ED., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain saturated hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene,” as used herein, refers to a divalent group derived from a straight or branched saturated chain hydrocarbon, for example, of 1 to 6 carbon atoms. Representative examples of alkylene include, but are not limited to, CH₂—, —CD₂—, —CH₂CH₂—, —CH₂CH₂CH₂—, —CH₂CH₂CH₂CH₂—, and —CH₂CH₂CH₂CH₂CH₂—.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Abbreviations: DCM=dichloromethane; equiv=equivalent(s); h=hour(s); min=minute(s); TEA=triethylamine; THF=tetrahydrofuran.

### 2. Polymyxins, Adjuvant Compounds, and Methods of Use

Polymyxin antibiotics are well known in the art, as described in Velkov T, Thompson P E, Nation R L, Li J. Structure—activity relationships of polymyxin antibiotics. J. Med. Chem. 2010; 53(5):1898-1916; de Visser P C, Kriek N M, van Hooft P A, et al. Solid-phase synthesis of polymyxin B1 and analogues via a safety-catch approach. J. Pept. Res. 2003; 61(6):298-306.Okimura K, Ohki K, Sato Y, Ohnishi K, Sakura N. Semi-synthesis of polymyxin B (2-10) and colistin (2-10) analogs employing the trichloroethoxycarbonyl (Troc) group for side chain protection of alpha, gamma-diaminobutyric acid residues. Chem. Pharm. Bull. (Tokyo). 2007; 55(12):1724-1730; Sakura N, Itoh T, Uchida Y, et al. The contribution of the N-terminal structure of polymyxin B peptides to antimicrobial and lipopolysaccharide binding activity. Bull. Chem. Soc. Jpn. 2004; 77(10):1915-1924; Chihara S, Ito A, Yahata M, Tobita T, Koyama Y. Chemical synthesis, isolation and characterization of α-N-fatty acyl colistin nonapeptide with special reference to the correlation between antimicrobial activity and carbon number of fatty acyl moiety. Agr. Biol. Chem. 1974; 38(3):521-529; Tsubery H, Ofek I, Cohen S, Fridkin M. N-terminal modifications of polymyxin B nonapeptide and their effect on antibacterial activity. Peptides. 2001; 22(10):1675-1681; Vaara M. The outer membrane permeability-increasing action of linear analogues of polymyxin B nonapeptide. Drugs Exp. Clin. Res. 1991; 17(9):437-443. Quale J, Shah N, Kelly P, et al. Activity of polymyxin B and the novel polymyxin analogue CB-182,804 against contemporary Gram-negative pathogens in New York City. Microb. Drug Resist. 2012; 18(2):132-136; Okimura K, Ohki K, Sato Y, Ohnishi K, Uchida Y, Sakura N. Chemical conversion of natural polymyxin B and colistin to their N-terminal derivatives. Bull. Chem. Soc. Jpn. 2007; 80(3):543-552; Katsuma N, Sato Y, Ohki K, Okimura K, Ohnishi K, Sakura N. Development of des-fatty acylpolymyxin B decapeptide analogs with Pseudomonas aeruginosa-specific antimicrobial activity. Chem. Pharm. Bull. (Tokyo). 2009; 57(4):332-336; Barnett M, Bushby S R, Wilkinson S. Sodium sulphomethyl derivatives of polymyxins. Br. J. Pharmacol. Chemother. 1964; 23:552-574; Vaara M, Fox J, Loidl G, et al. Novel polymyxin derivatives carrying only three positive charges are effective antibacterial agents. Antimicrob. Agents Chemother. 2008; 52(9):3229-3236; Kimura Y, Matsunaga H, Vaara M. Polymyxin B octapeptide and polymyxin B heptapeptide are potent outer membrane permeability-increasing agents. J. Antibiot. (Tokyo). 1992; 45(5):742-749; Kanazawa K, Sato Y, Ohki K, et al. Contribution of each amino acid residue in polymyxin B(3) to antimicrobial and lipopolysaccharide binding activity. Chem. Pharm. Bull. (Tokyo). 2009; 57(3): 240-244. Tsubery H, Ofek I, Cohen S, Fridkin M. Structure activity relationship study of polymyxin B nonapeptide. Adv. Exp. Med. Biol. 2000; 479:219-222, which are incorporated herein by reference.

Polymyxin antibiotics include those produced in nature by gram-positive bacteria (e.g., Paenibacillus polymyxa), such as polymyxins B and E, as well as synthetic polymyxins. Polymyxins typically contain 10 amino acid residues, six of which are L-α,γ-diaminobutyric acid (L-DAB). The DAB residues cause polymyxins to have multiple positively charged groups at physiological pH. Seven amino acid residues form the main cyclic component (with hydrophobic residues at positions 6 and 7) while the other three extend from one of the cyclic residues as a linear chain terminating in an N-terminal fatty acyl group (e.g., 6-methyloctanoic acid, 6-methylheptanoic acid). The amino acid residues and DAB monomers are generally in the L (levo) configuration, however certain strains such as P. polymyxa PKB1 have been observed to incorporate DAB with the D (dextro) configuration at position 3 producing variations of polymyxin B. Polymyxin M is also known as “mattacin”.

Polymyxins B and E include the following known structures.

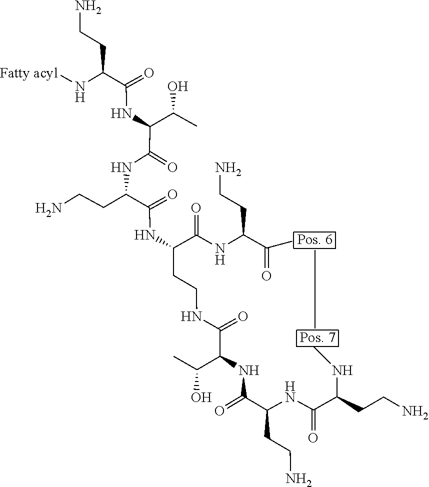

| Polymyxin | Fatty acyl group | Pos. 6 | Pos. 7 |
|---|---|---|---|
| B1 | (S)-6-methyloctanoyl | D-Phe | Leu |
| Bl-Ile | (S)-6-methyloctanoyl | D-Phe | Ile |
| B2 | 6-methylheptanoyl | D-Phe | Leu |
| B3 | Octanoyl | D-Phe | Leu |
| B4 | Heptanoyl | D-Phe | Leu |
| B5 | Nonanoyl | D-Phe | Leu |
| B6 | 3-hydroxy-6-methyloctanoyl | D-Phe | Leu |
| E1 | (S)-6-methyloctanoyl | D-Leu | Leu |
| E2 | 6-methylheptanoyl | D-Leu | Leu |
| E3 | Octanoyl | D-Leu | Leu |
| E4 | Heptanoyl | D-Leu | Leu |
| E7 | 7-methyloctanoyl | D-Leu | Leu |
| E1-Ile | (S)-6-methyloctanoyl | D-Leu | Ile |
| E1-Val | (S)-6-methyloctanoyl | D-Leu | Val |
| E1-Nva | (S)-6-methyloctanoyl | D-Leu | Nva |
| E2-Ile | 6-methylheptanoyl | D-Leu | Ile |
| E2-Val | 6-methylheptanoyl | D-Leu | Val |
| E8-Ile | 7-methylnonanoyl | D-Leu | Ile |

Colistin is a polymyxin E and available in a form which can be injected into a vein or muscle or inhaled, known as colistimethate sodium and one which is applied to the skin or taken by mouth, known as colistin sulfate. Polymyxin B is administered directly as an active antibiotic.

Two forms of colistin are available commercially: colistin sulfate and colistimethate sodium (colistin methanesulfonate sodium, colistin sulfomethate sodium). Colistin sulfate is cationic; colistimethate sodium is anionic. Colistin sulfate is stable, but colistimethate sodium is readily hydrolysed to a variety of methanesulfonated derivatives. Colistin sulfate and colistimethate sodium are eliminated from the body by different routes. With respect to Pseudomonas aeruginosa, colistimethate is the inactive prodrug of colistin.

Colistimethate sodium may be administered by injection or inhaled. Colistimethate sodium may be used to treat Pseudomonas aeruginosa infections in cystic fibrosis patients, and in treating multidrug-resistant Acinetobacter infection. Colistimethate sodium has been given intrathecally and intraventricularly in Acinetobacter baumannii and Pseudomonas aeruginosa meningitis/ventriculitis. Colistin may be useful for treating infections caused by carbapenem-resistant isolates of Acinetobacter baumannii.

Colistin sulfate may be used to treat intestinal infections, or to suppress colonic flora. Colistin sulfate can be administered orally as tablets and syrup for selective digestive tract decontamination (no absorption) and topically for the treatment of bacterial skin infections (e.g., in the form of topical creams, powders, and otic solutions).

Colistin sulfate and colistimethate sodium may both be given intravenously Suitable injectable doses of colistamethate sodium include doses from 200 to 800 mg/day.

The dosage of intravenous colistin recommended by the manufacturers in the United States is 2.5-5 mg/kg (31,250-62,500 IU/kg) per day, divided into 2-4 equal doses (1 mg of colistin equals 12,500 IU) (This dosage refers to adult patients with normal renal function Coly-mycin M parenteral [package insert]. Bristol, Tenn.: Monarch Pharmaceuticals, 2002). The dosage recommended by the manufacturers in the United Kingdom is 4-6 mg/kg (50,000-75,000 IU/kg) per day, in 3 divided doses for adults and children with body weights of ≤60 kg and 80-160 mg (1-2 million IU) every 8 h for those with body weights of >60 kg (Colomycin [package insert]. Bexley, UK: Forest Laboratories, UK Limited, 2002). However, patients may be treated with higher daily doses of colistin administered intravenously, up to 720 mg (9 million IU) per day (in 3 divided doses) (Michalopoulos et al., Clin Microbiol Infect 2005; 11:115-21; Markou et al., Crit Care 2003; 7:R78-83). Modifications of the total daily dose are required in the presence of renal impairment.

Besides the intermittent intravenous mode of administration, colistin can also be administered by continuous 24-h infusion (Michalopoulos et al., Scand J Infect Dis 2005 37:142-5). In addition, colistin can be used intramuscularly at the same doses recommended for intravenous administration. However, intramuscular administration is not commonly used in clinical practice because of the severe pain caused at the injection site. In addition, polymyxin B with a caine-type local anesthetic is a combination permitted by the US Food and Drug Administration (FDA) that can be administered intramuscularly, in ear drops, and in ointments.

When colistin is given by inhalation, the dosage recommended by the manufacturers in the United Kingdom is 40 mg (500,000 IU) every 12 h for patients with body weights of ≤40 kg and 80 mg (1 million IU) every 12 h for patients with body weights of >40 kg. For recurrent pulmonary infections, the dosage of aerosolized colistin can be increased to 160 mg (2 million IU) every 8 h (Promixin 1 MIU powder for nebuliser solution [package insert].West Sussex, UK: Profile Pharma Limited, 2003). For spontaneously breathing patients, colistin can be administered as follows: 80 mg (1 million IU) of colistin is added to 4 mL of normal saline and swirled slowly to mix, and the solution is nebulized with 8 L/min oxygen flow and inhaled via a face mask. For patients undergoing mechanical ventilation, aerosolized colistin can be delivered by means of most ventilators. In addition, usually for patients with cystic fibrosis, inhaled colistin can be administered through jet or ultrasonic nebulizers (Weber et al., Pediatr Pulmonol 1997; 23:249-60; Faurisson et al. Respiration 1995; 62(Suppl 1):13-8).

The dosage of colistin used in cases for intrathecal administration ranged from 3.2 mg (40,000 IU) to 10 mg (125,000 IU) given once per day and in 2 cases for intraventricular administration ranged from 10 mg (125,000 IU) to 20 mg (250,000 IU) per day (divided into 2 doses). In these cases, no additional intravenous colistin was administered (Benifla et al., J Antimicrob Chemother 2004; 54:290-2; Fernandez-Viladrich et al., Clin Infect Dis 1999; 28:916-7; Vasen et al., J Clin Microbiol 2000; 38:3523).

Colistin may be used in combination with rifampicin, and evidence of in-vitro synergy exists and the combination has been used successfully in patients. There is also in-vitro evidence of synergy for colistimethate sodium used in combination with other antipseudomonal antibiotics.

Colistimethate sodium aerosol (Promixin; Colomycin Injection) may be used to treat pulmonary infections, especially in cystic fibrosis. A recommended adult dose is 1-2 million units (80-160 mg) nebulized colistimethate twice daily. Nebulized colistin has also been used to decrease severe exacerbations in patients with chronic obstructive pulmonary disease and infection with Pseudomonas aeruginosa.

In the compounds of formula (I) used in the therapeutic methods/uses described herein, formula (I) may have formula (II)

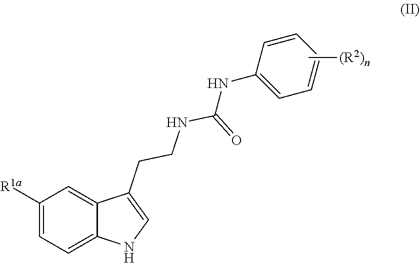

wherein R^{1a} is hydrogen, halogen, cyano, CH₃, OCH₃, OCF₃, or OCHF₂; R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, or —OC₃₋₆cycloalkyl; and n is 0, 1, 2, 3, 4, or 5.

Formula (II), in turn, may have any of the following formulas:

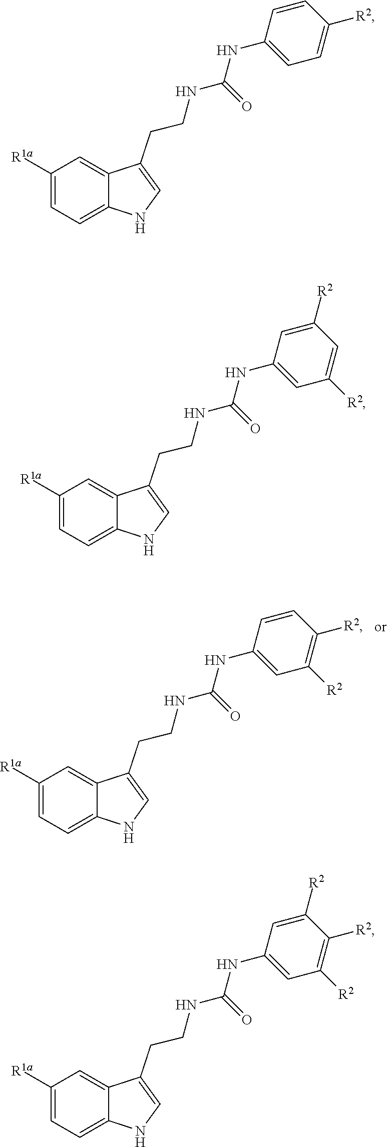

wherein R^{1a} and R² are as defined herein.

Compounds of formula (II) may have any of the following formulas:

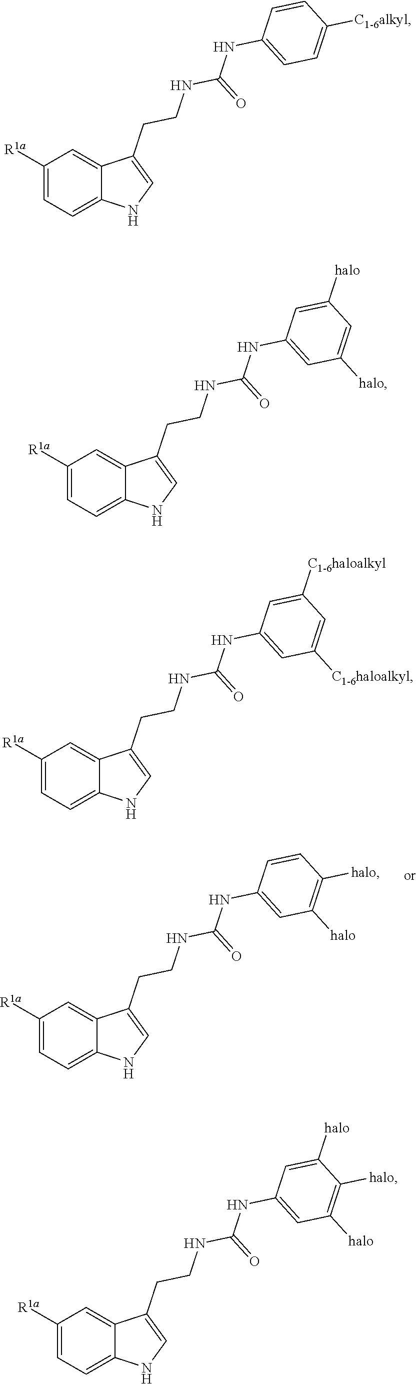

wherein R^{1a} is as defined herein.

Compounds of formula (II) may have any of the following formulas:

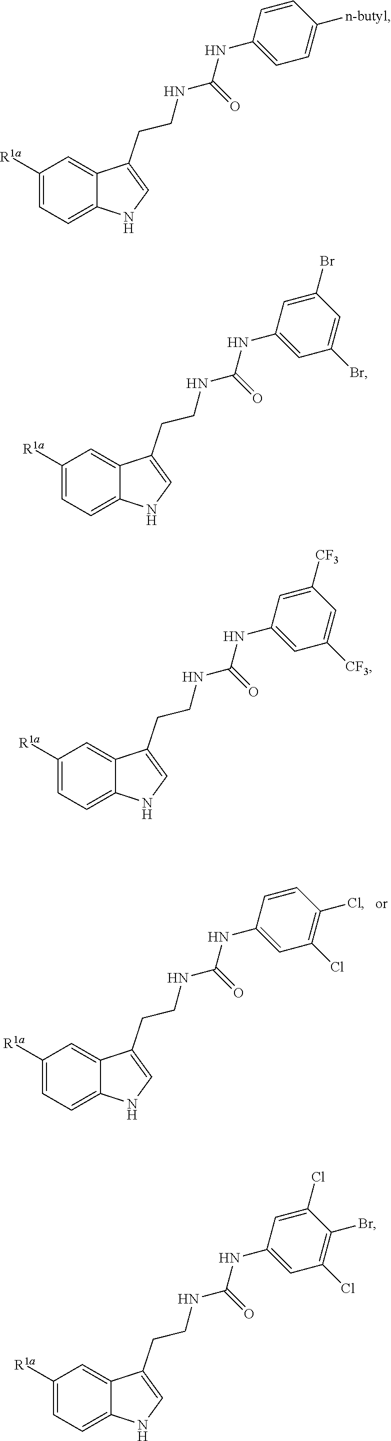

wherein R^{1a} is as defined herein.

In any of the compounds of formula (II), R^{1a} may be hydrogen. In any of the compounds of formula (II), R^{1a} may be halogen (e.g., bromo).

In the compounds of formula (I) used in the therapeutic methods/uses described herein, formula (I) may have formula (III)

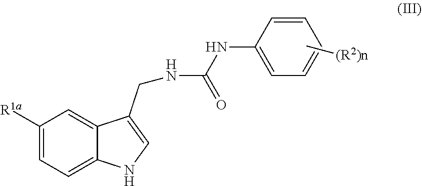

wherein R^{1a} is hydrogen, halogen, cyano, CH₃, OCH₃, OCF₃, or OCHF₂; R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, or —OC₃₋₆cycloalkyl; and n is 0, 1, 2, 3, 4, or 5.

Formula (III), in turn, may have any of the following formulas:

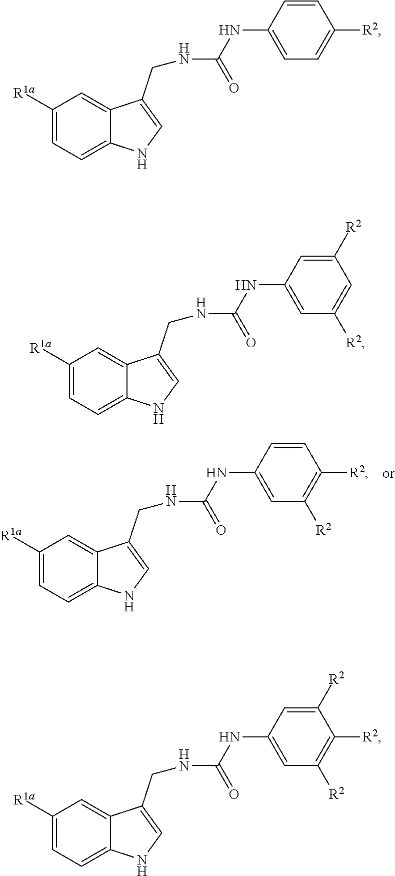

wherein R^{1a} and R² are as defined herein.

Compounds of formula (III) may have any of the following formulas:

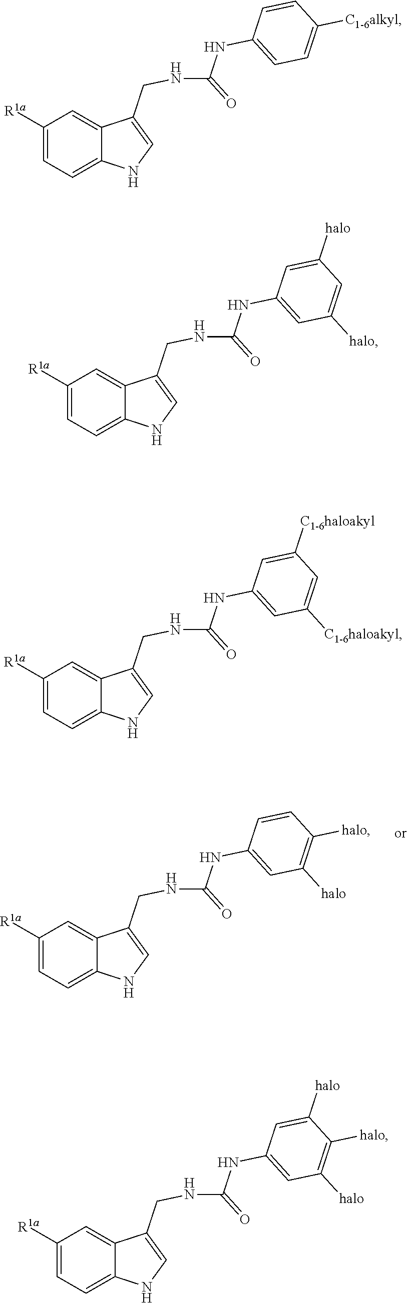

wherein R^{1a} is as defined herein.

Compounds of formula (III) may have any of the following formulas:

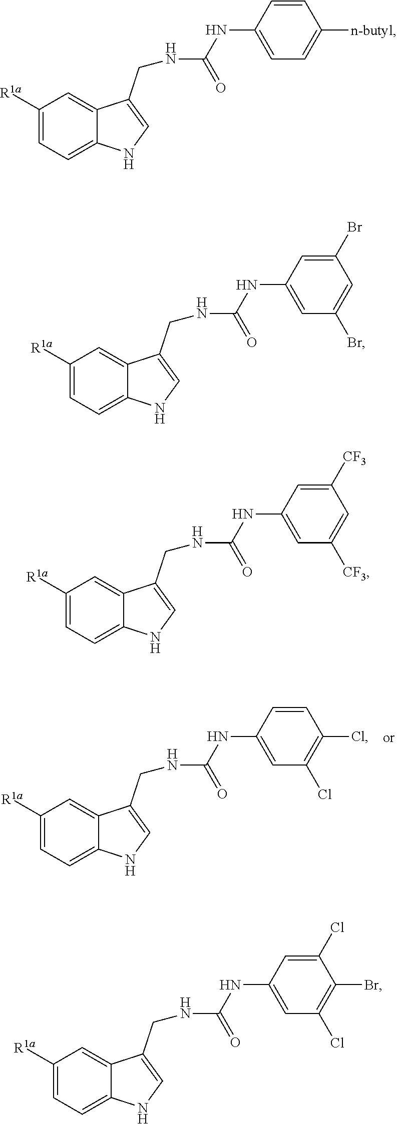

wherein R^{1a} is as defined herein.

In any of the compounds of formula (III), R^{1a} may be hydrogen. In any of the compounds of formula (III), R^{1a} may be halogen (e.g., bromo).

In the compounds of formula (I) used in the therapeutic methods/uses described herein, formula (I) may have formula (IV)

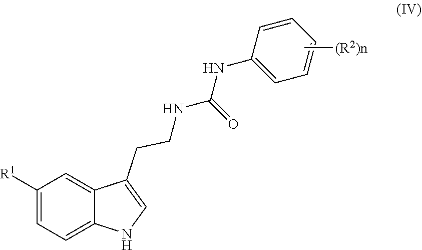

wherein R¹ is halogen, cyano, CH₃, OCH₃, OCF₃, or OCHF₂; R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, —OC₃₋₆cycloalkyl; and n is 0, 1, 2, 3, 4, or 5. Preferably, the compound of formula (IV) is not:
N-[2-(5-fluoro-1H-indol-3-yl)ethyl]-N′-(4-methoxyphenyl)-urea;
N-(2-fluorophenyl)-N′-[2-(5-methyl-1Hindol-3-yl)ethyl]-urea;
N-(3-fluorophenyl)-N′-[2-(5-methyl-1Hindol-3-yl)ethyl]-urea;
N-(4-ethoxyphenyl)-N′-[2-(6-methoxy-1Hindol-3-yl)ethyl]-urea;
N-[2-(5-chloro-1H-indol-3-yl)ethyl]-N′-(2,4-dimethoxyphenyl)-urea;
N-(3,5-dimethoxyphenyl)-N′-[2-(5-fluoro-1H-indol-3-yl)ethyl]-urea;
N-[2-(5-fluoro-1H-indol-3-yl)ethyl]-N′-(3-methylphenyl)-urea;
N-(5-chloro-2-methoxyphenyl)-N′-[2-(5-fluoro-1H-indol-3-yl)ethyl]-urea;
N-(3-bromophenyl)-N′-[2-(5-fluoro-1Hindol-3-yl)ethyl]-urea;
N-(3-chlorophenyl)-N′-[2-(5-fluoro-1Hindol-3-yl)ethyl]-urea;
N-(4-chlorophenyl)-N′-[2-(5-fluoro-1Hindol-3-yl)ethyl]-urea;
N-(3,4-dichlorophenyl)-N′-[2-(5-fluoro-1Hindol-3-yl)ethyl]-urea;
N-[2-(5-fluoro-1H-indol-3-yl)ethyl]-N′-(4-methylphenyl)-urea;
N-[2-(5-fluoro-1H-indol-3-yl)ethyl]-N′-phenyl-urea;
N-[2-(5-chloro-1H-indol-3-yl)ethyl]-N′-(4-methylphenyl)-urea;
N-[2-(5-methyl-1H-indol-3-yl)ethyl]-N′-(4-methylphenyl)-urea;
N-[2-(5-chloro-1H-indol-3-yl)ethyl]-N′-(4-fluorophenyl)-urea;
N-[2-(5-chloro-1H-indol-3-yl)ethyl]-N′-(4-nitrophenyl)-urea;
N-[2-(5-chloro-1H-indol-3-yl)ethyl]-N′-(3-chlorophenyl)-urea;
N-[2-(5-chloro-1H-indol-3-yl)ethyl]-N′-(3,4-dichlorophenyl)-urea;
N-[2-(5-chloro-1H-indol-3-yl)ethyl]-N′-(4-chlorophenyl)-urea;
N-[2-(5-chloro-1H-indol-3-yl)ethyl]-N′-phenyl-urea;
N-(4-fluorophenyl)-N′-[2-(5-methyl-1Hindol-3-yl)ethyl]-urea;
N-[2-(5-methyl-1H-indol-3-yl)ethyl]-N′-(4-nitrophenyl)-urea;
N-(3-chlorophenyl)-N′-[2-(5-methyl-1Hindol-3-yl)ethyl]-urea;
N-(3,4-dichlorophenyl)-N′-[2-(5-methyl-1Hindol-3-yl)ethyl]-urea;
N-(4-chlorophenyl)-N′-[2-(5-methyl-1Hindol-3-yl)ethyl]-urea; or
N-[2-(5-methyl-1H-indol-3-yl)ethyl]-N′-phenyl-urea.

Formula (IV), in turn, may have any of the following formulas:

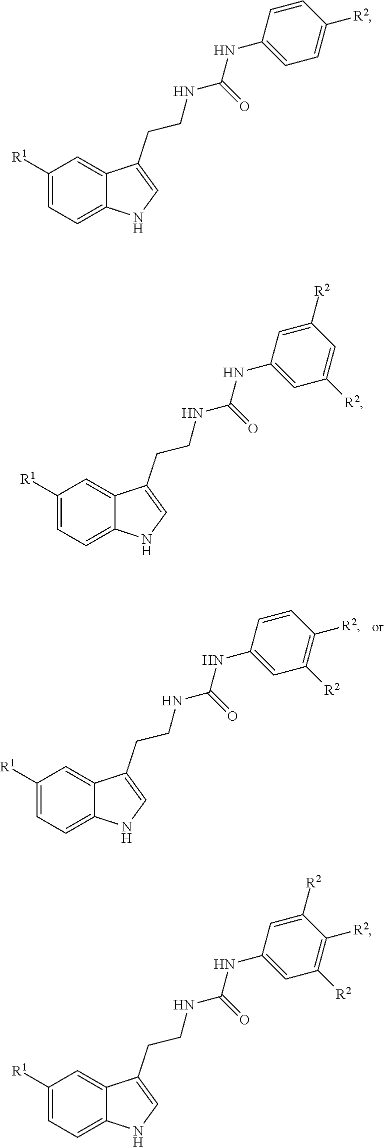

wherein R¹ and R² are as defined herein.

Compounds of formula (IV) may have any of the following formulas:

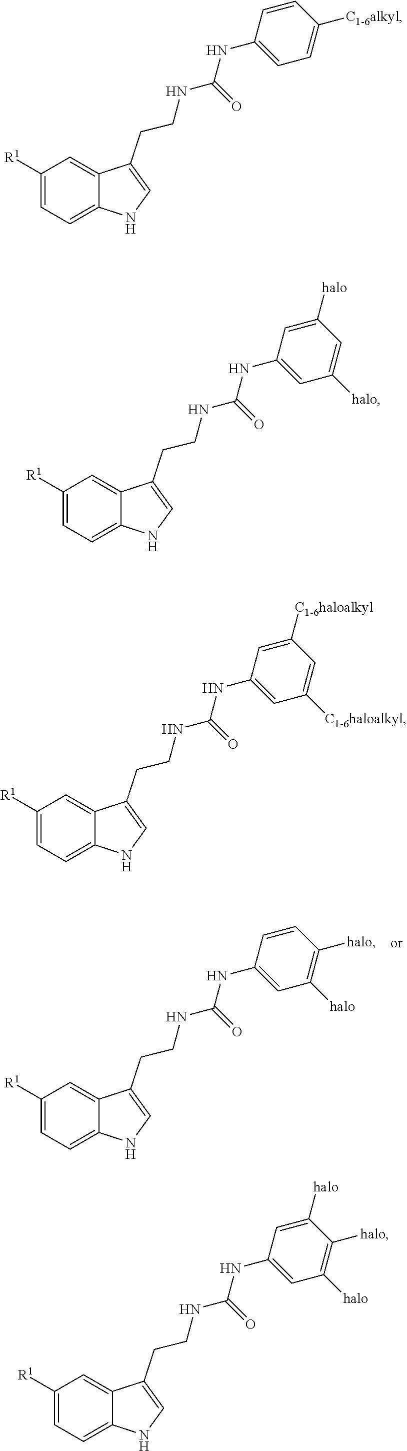

wherein R¹ is as defined herein.

Compounds of formula (IV) may have any of the following formulas:

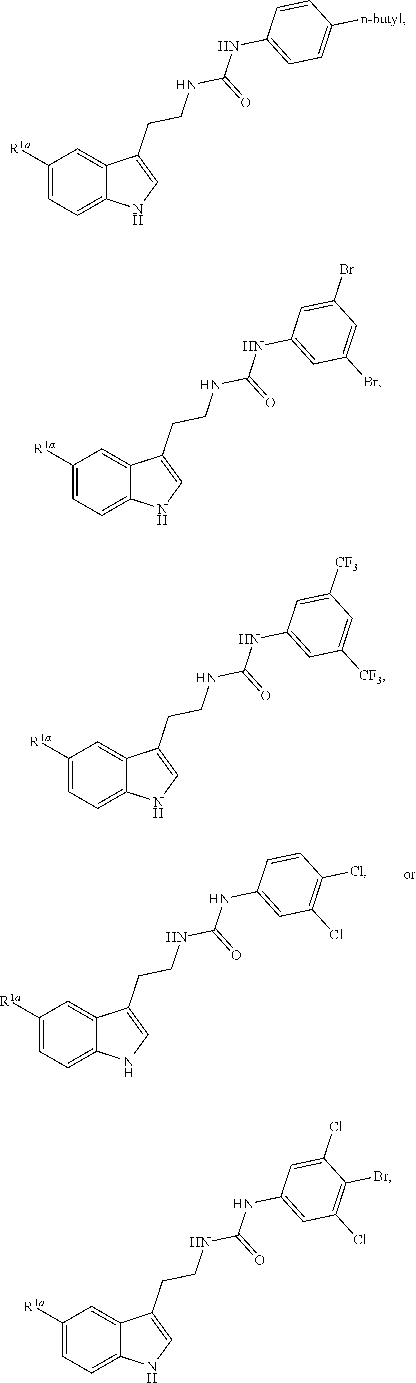

wherein R¹ is as defined herein.

Throughout the embodiments and description of the compounds of the invention, all instances of haloalkyl may be fluoroalkyl (e.g., any C₁₋₄haloalkyl may be C₁₋₄fluoroalkyl).

The compound of formula (I) may be selected from the group consisting of:
1-(2-(1H-indol-3-yl)ethyl)-3-(4-butylphenyl)urea;
1-(2-(1H-indol-3-yl)ethyl)-3-(3,5-dibromophenyl)urea;
1-(2-(1H-indol-3-yl)ethyl)-3-(3,5-bis(trifluoromethyl)phenyl)urea;
1-(2-(1H-indol-3-yl)ethyl)-3-(3,4-dichlorophenyl)urea;
1-(2-(1H-indol-3-yl)ethyl)-3-(4-bromo-3,5-dichlorophenyl)urea;
1-(2-(5-bromo-1H-indol-3-yl)ethyl)-3-(4-butylphenyl)urea;
1-(2-(5-bromo-1H-indol-3-yl)ethyl)-3-(3,5-dibromophenyl)urea;
1-(3,5-bis(trifluoromethyl)phenyl)-3-(2-(5-bromo-1H-indol-3-yl)ethyl)urea;
1-(2-(5-bromo-1H-indol-3-yl)ethyl)-3-(3,4-dichlorophenyl)urea;
1-(2-(5-bromo-1H-indol-3-yl)ethyl)-3-(4-bromo-3,5-dichlorophenyl)urea;
1((1H-indol-3-yl)methyl)-3-(4-butylphenyl)urea;
1((1H-indol-3-yl)methyl)-3-(3,5-dibromophenyl)urea;
1((1H-indol-3-yl)methyl)-3-(3,5-bis(trifluoromethyl)phenyl)urea;
1((1H-indol-3-yl)methyl)-3-(3,4-dichlorophenyl)urea; and
1((1H-indol-3-yl)methyl)-3-(4-bromo-3,5-dichlorophenyl)urea.

The compound of formula (I) may be selected from the group consisting of:
1-(2-(1H-indol-3-yl)ethyl)-3-(3,5-dibromophenyl)urea;
1-(2-(1H-indol-3-yl)ethyl)-3-(4-bromo-3,5-dichlorophenyl)urea;
1-(2-(5-bromo-1H-indol-3-yl)ethyl)-3-(4-butylphenyl)urea;
1-(2-(5-bromo-1H-indol-3-yl)ethyl)-3-(3,5-dibromophenyl)urea;
1-(3,5-bis(trifluoromethyl)phenyl)-3-(2-(5-bromo-1H-indol-3-yl)ethyl)urea;
1-(2-(5-bromo-1H-indol-3-yl)ethyl)-3-(3,4-dichlorophenyl)urea;
1-(2-(5-bromo-1H-indol-3-yl)ethyl)-3-(4-bromo-3,5-dichlorophenyl)urea;
1((1H-indol-3-yl)methyl)-3-(4-butylphenyl)urea;
1((1H-indol-3-yl)methyl)-3-(3,5-dibromophenyl)urea;
1((1H-indol-3-yl)methyl)-3-(3,5-bis(trifluoromethyl)phenyl)urea;
1((1H-indol-3-yl)methyl)-3-(3,4-dichlorophenyl)urea; and
1((1H-indol-3-yl)methyl)-3-(4-bromo-3,5-dichlorophenyl)urea.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

Compounds may possess tautomeric forms, as well as geometric isomers, and that these also constitute embodiments of the disclosure.

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

In one aspect, the invention provides methods of treating a gram-negative bacterial infection comprising administering to a subject in need thereof, a polymyxin antibiotic, or a pharmaceutically acceptable salt thereof, and a compound of formula (I) in amounts that together are effective to inhibit the gram-negative bacterial infection.

In another aspect, the invention provides a method of potentiating the activity of a polymyxin antibiotic against a gram-negative bacterial infection comprising administering to a subject in need thereof a compound of formula (I) in an amount effective to increase the therapeutic effect of a dose of the polymyxin antibiotic, or a pharmaceutically acceptable salt thereof, compared to the therapeutic effect of the dose of the polymyxin antibiotic, or a pharmaceutically acceptable salt thereof, in the absence of the compound of formula (I).

In another aspect, the invention provides a use of a pharmaceutical combination of a polymyxin antibiotic, or a pharmaceutically acceptable salt thereof, and a compound of formula (I) for the preparation of a medicament for the treatment of a gram-negative bacterial infection.

In another aspect, the invention provides a use of a pharmaceutical combination of a polymyxin antibiotic, or a pharmaceutically acceptable salt thereof, and a compound of formula (I) for the preparation of a medicament for potentiation of the activity of a polymyxin antibiotic, or a pharmaceutically acceptable salt thereof, against a gram-negative bacterial infection.

In the methods and uses described herein, the gram-negative bacterial infection may be a multidrug-resistant gram-negative bacterial infection. Multidrug-resistant gram-negative bacteria are a type of Gram-negative bacteria with resistance to multiple antibiotics. Gram-negative bacteria can acquire resistance to one or more classes of antibiotics such as Ureidopenicillins (piperacillin), cephalosporins (cefotaxime, ceftazidime), carbapenems (imipenem, meropenem), fluorquinolones (ciprofloxacin), polymyxins (colistin and polymyxin B), aminoglycosides (gentamicin, amikacin), glycylcycline (tigecycline), tetracyclines (doxycycline, minocycline), chloramphenicol, sulphonamides (co-trimoxazole), and fosfomycin. As used herein “multi-drug resistant” means acquired non-susceptibility to at least one agent in three or more antimicrobial categories.

The gram-negative bacterial infection may be an infection of a bacteria species of the Enterobacteriaceae family, including Escherichia coli, Enterobacter spp., Klebsiella spp., Citrobacter spp., Salmonella spp., and Shigella spp. Other gram-negative bacteria include Acinetobacter baumannii, Pseudomonas aeruginosa, and Stenotrophomonas maltophilia. The gram-negative bacteria may be selected from one or more of E. coli, K. pneumonia, and A. baumannii.

The gram-negative bacterial infection may be an infection of a bacterial strain possessing a mobilized colistin resistance (mcr) gene, which includes mcr-1, mcr-2, mcr-3, mcr-4, mcr-5, mcr-6, mcr-7, mcr-8, mcr-9, or mcr-10, as described in Carroll et al., mBio (2019) 10(3), DOI: 10.1128/mBio.00853-19 and Wang et al., Emerging Microbes & Infections (2020) 9:1, 508-516, which are incorporated herein by reference.

Compounds of formula (I), (II), (III), or (IV) are adjuvants that may sensitize gram-negative bacteria to the effects of a polymyxin antibiotic, but are otherwise without appreciable antibiotic activity alone. The gram-negative bacteria may or may not be a polymyxin-resistant bacteria, i.e., resistant to the antibiotic effects of a polymyxin. In some embodiments, the bacteria is resistant to colistin and may be sensitized to the effects of colistin by compounds of formula (I), (II), (III), or (IV). Colistin resistance may be encoded in the mcr-1 gene.

Sensitization of resistant bacteria refers to an adjuvant effect of rendering effective (i.e., inhibitory) a non-effective or non-inhibitory amount (concentration or dose) of the polymyxin. Sensitization also includes increasing the effectiveness of an amount of the polymyxin that is sub-optimally effective against a bacterium. Sensitization includes rendering a polymyxin antibiotic (e.g., colistin in whichever form) effective against a colistin-resistant bacterium at doses of the polymyxin that would be effective against a normally polymyxin-sensistive bacterium, in the absence of adjuvant. In either case, the adjuvant potentiates the antibacterial effect of the polymyxin. The overall effect of sensitization is to lower the effective dose or concentration of the polymyxin against the bacteria. The improved effectiveness may be measured in terms of a reduced inhibitory concentration or reduced therapeutically effective concentration or dose of polymyxin compared to the polymyxin dosed without the adjuvant. For example, the improved effectiveness may be a reduction in minimum inhibitory concentration, minimum therapeutically effective dose, or ED₅₀.

The polymyxin antibiotic and compound of formula (I), (II), (III), or (IV) are administered in amounts that together are more effective to inhibit a gram-negative bacteria than administration of the same amount of the polymyxin without the compound of formula (I), (II), (III), or (IV). A therapeutically effective dose/amount of a compound of formula (I), (II), (III), or (IV) sensitizes a gram-negative bacteria to the effects of a polymyxin antibiotic. A therapeutically effective dose/amount of a compound of formula (I), (II), (III), or (IV) may increase the antibacterial effect of the polymyxin by reducing the polymyxin minimum effective concentration/dose/amount by from 1.5 to over 2000-fold or more, depending on the bacterial strain.

As used herein, the term “treat” or “treating” a subject having a disorder refers to administering a compound or a composition described herein to the subject, such that at least one symptom of the disorder is cured, healed, alleviated, relieved, altered, remedied, ameliorated, or improved. Treating includes administering an amount effective to alleviate, relieve, alter, remedy, ameliorate, cure, improve or affect the disorder or the symptoms of the disorder. The treatment may inhibit deterioration or worsening of a symptom of a disorder.

Doses may include a “therapeutically effective amount” of an agent. A “therapeutically effective amount” refers to sufficient amounts of the compounds to treat disorders, at a reasonable benefit/risk ratio applicable to any medical treatment. It is understood, however, that the total daily dosage of the compounds and compositions can be decided by the attending physician within the scope of sound medical judgment. The specific therapeutically effective dose level for any particular patient can depend upon a variety of factors including the disorder being treated and the severity of the disorder; activity of the specific compound employed; the specific composition employed; the age, body weight, general health and prior medical history, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved. Actual dosage levels of active ingredients in the pharmaceutical compositions can be varied so as to obtain an amount of the active compound(s) that is effective to achieve the desired therapeutic response for a particular patient and a particular mode of administration. In the treatment of certain medical conditions, repeated or chronic administration of compounds can be required to achieve the desired therapeutic response. “Repeated or chronic administration” refers to the administration of compounds daily (i.e., every day) or intermittently (i.e., not every day) over a period of days, weeks, months, or longer.

For example, a therapeutically effective amount of a compound of formula (I), (II), (III), or (IV) may be about 0.1 mg/kg to about 1000 mg/kg, about 0.5 mg/kg to about 1000 mg/kg, about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

In the methods and uses described herein, the polymyxin antibiotic and the compound of formula (I), (II), (III), or (IV) (pharmaceutical combination) may be administered/used simultaneously, separately, or sequentially, and in any order, and the components may be administered separately or as a fixed combination. For example, the delay of progression or treatment of diseases according to the invention may comprise administration of the first active ingredient in free or pharmaceutically acceptable salt form and administration of the second active ingredient in free or pharmaceutically acceptable salt form, simultaneously or sequentially in any order, in jointly therapeutically effective amounts or effective amounts, e.g. in daily dosages. The individual active ingredients of the combination can be administered separately at different times during the course of therapy or concurrently in divided or single dosage forms, and by the same or different routes of administration. The instant invention is therefore to be understood as embracing all such regimes of simultaneous or alternating treatment and the term “administering” is to be interpreted accordingly. Thus, a pharmaceutical combination, as used herein, defines either a fixed combination in one dosage unit form or separate dosage forms for the combined administration where the combined administration may be independently at the same time or at different times.

In another aspect, the therapeutic treatment methods described herein may further include administration of one or more additional antibiotics from the classes such as ureidopenicillins (e.g., piperacillin), cephalosporins (e.g., cefotaxime, ceftazidime, ceftobiprole, ceftaroline, FR-264205), carbapenems (e.g., imipenem, meropenem), fluorquinolones (e.g., ciprofloxacin), aminoglycosides (e.g., gentamicin, amikacin), glycylcycline (e.g., tigecycline), tetracyclines (e.g., doxycycline, minocycline), chloramphenicol, sulphonamides (e.g., co-trimoxazole), and fosfomycin.

In another aspect, the disclosure provides a kit comprising at least one disclosed compound of formula (I), (II), (III), or (IV); at least one polymyxin (e.g., colisin); and optionally an additional gram-negative antibiotic. The therapeutic agents of the kit may be in the form of a pharmaceutical composition. Kits may further include instructions for administering the therapeutic agents. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient. That the disclosed kits can be employed in connection with disclosed methods of use. The kits may further comprise information, instructions, or both that use of the kit will provide treatment for medical conditions in mammals (particularly humans). The information and instructions may be in the form of words, pictures, or both, and the like. In addition or in the alternative, the kit may include the compound, a composition, or both; and information, instructions, or both, regarding methods of application of compound, or of composition, preferably with the benefit of treating or preventing medical conditions in mammals (e.g., humans).

### 3. General Synthesis

The compounds of the present disclosure can be prepared in a number of ways well known to one skilled in the art of organic synthesis. The compounds of the present disclosure can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated in their entirety by reference as to the subject matter referenced herein. Compounds of formula (I) may be also prepared by metabolic processes. Preparation of the compounds by metabolic processes includes those occurring in the human or animal body (in vivo) or processes occurring in vitro.

The compounds of the disclosure may be prepared using the exemplary reactions and techniques described in this section. The reactions are performed in solvents appropriate to the reagents and materials employed and are suitable for the transformations being effective. Also, in the description of the synthetic methods described below, it is to be understood that all proposed reaction conditions, including solvent, reaction atmosphere, reaction temperature, duration of the experiment, and workup procedures, are chosen to be the conditions standard for that reaction, which should be readily recognized by one skilled in the art. One having ordinary skill in the art may adjust one or more of the conditions described herein. One skilled in the art of organic synthesis understands that the functionality present on various portions of the edict molecule must be compatible with the reagents and reactions proposed. Not all compounds of the disclosure falling into a given class may be compatible with some of the reaction conditions required in some of the methods described. Such restrictions to the substituents, which are compatible with the reaction conditions, will be readily apparent to one skilled in the art and alternate methods can be used.

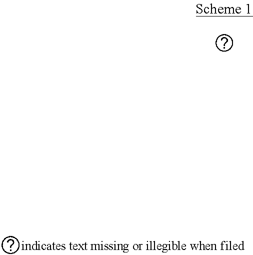

Amide compounds 1a-d and 6a-d may be prepared as shown in Scheme 1 by reaction of the appropriate tryptamine 5 with an aroyl chloride in the presence of a base, such as triethylamine in an organic solvent, such as dichloromethane.

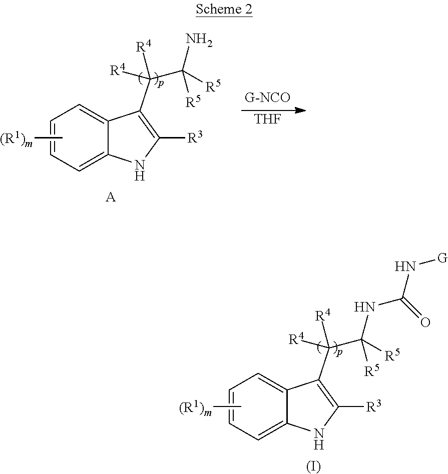

Compounds of formula (I) may be prepared as shown in Scheme 2 by reaction of amine A with an isocyante G-NCO.

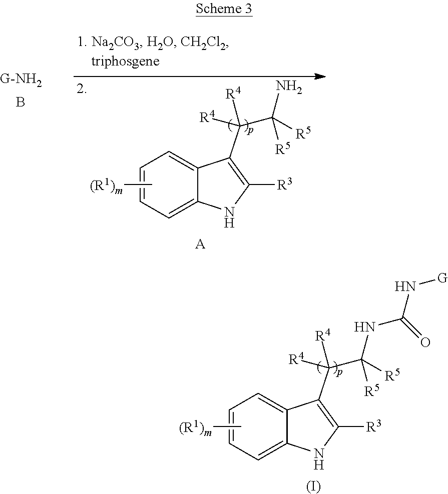

In some cases, compounds of formula (I) may be prepared as shown in Scheme 3 by first reacting an aniline B with triphosgene to form an intermediate isocyanate, followed by reaction with the amine A.

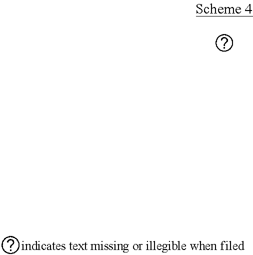

As more specifically illustrated in Scheme 4, typtamine 5 may be reacted with a commercially available isocyanate 7 to provide compounds 8a, 8d, 9a, or 9d. Alternatively, commercial anilines (route B) may be reacted with triphosgene to form an intermediate isocyanate, followed by reaction with the tryptamine 5 to provide compounds 8b, 8c, 8e, 9b, 9c, or 9e.

Compounds 11a-e, 12a-e, and 13a-e may likewise be prepared by the foregoing methods.

### CHEMISTRY EXAMPLES

General. All reagents used for chemical synthesis were purchased from commercially available sources (VWR U.S. or Oakwood Chemical U.S.) and used without further purification. Flash chromatography was performed using 60 Å mesh standard grade silica gel from Sorbtech. NMR solvents were obtained from Cambridge Isotope Laboratories and used as is. ¹H NMR (400, 500 or 700 MHz) and ¹³C NMR (100, 125 or 175 MHz) spectra were recorded at 25° C. on Varian Mercury (700 MHz) or Bruker (500 or 400 MHz) spectrometers. Chemical shifts (δ) are given in parts per million relative to the respective NMR solvent; coupling constants (J) are in hertz (Hz). Abbreviations used are s, singlet; bs, broad singlet; d, doublet; dd, doublet of doublets; t, triplet; dt, doublet of triplets; m, multiplet. High resolution mass spectra were obtained at the ND Department of Chemistry Mass Spectrometry Facility. The purities of the tested compounds were all verified to be >95% by NMR and LC-MS analysis on an Advion LC-MS 2020 with Kinetex, 2.6 mm, C18 50×2.10 mm.

General procedure for amide formation (1a-e and 6a-e). In a flame dried round bottom flask under N₂ atmosphere was added tryptamine or 5-bromotryptamine (0.8 mmol, 1 equiv), TEA (0.35 mL, 2.5 mmol, 3 equiv), and anhydrous DCM (10 mL). The reaction mixture was cooled to 0° C. and the acid chloride (0.9 equiv, 0.75 mmol) was added dropwise. The reaction was stirred until completion as determined by TLC. Upon completion, the reaction was washed once with 1 M HCl (10 mL), once with brine (10 mL), and the organic layer was then dried with MgSO₄ and concentrated under reduced pressure. The crude product was then purified via flash chromatography (30% ethyl acetate/70% hexanes) to yield products 1a-e and 6a-e.

General procedure for urea formation via an isocyanate (aryl groups a and d). A solution of isocyanate (0.4 mmol, 2 equiv) dissolved in THF (10 mL) was added to a solution of indole (0.2 mmol, 1 equiv) in THF (10 mL). The reaction was stirred for 3 h. and then concentrated under reduced pressure. The crude product was purified via flash chromatography (20% ethyl acetate/80% hexanes).

General procedure for urea formation from aniline (phenyl rings b, c, and e). The desired aniline (0.2 mmol, 1 equiv) dissolved in DCM (12 mL) was added to a sodium carbonate (0.03 g, 0.39 mmol, 1.6 equiv) solution in H₂O (12 mL). After stirring for 5 min., triphosgene (0.03 g, 0.12 mmol, 0.33 equiv) was added to the flask. After stirring for 30 min., the indole (0.2 mmol, 1 equiv) was added dropwise and allowed to stir for an additional 2 h. The mixture was then separated and the aqueous layer was washed twice with DCM. The organic fractions were combined, dried with MgSO₄, and concentrated under reduced pressure. The crude product was then purified via flash chromatography (20% ethyl acetate/80% hexanes).

N-(2-(1H-indol-3-yl)ethyl)-3,5-dibromobenzamide (1b). Using the general procedure for amide formation with tryptamine and 3,5-dibromobenzoyl chloride, 1b was obtained as a white solid in 80% yield. ¹H NMR (500 MHz, Methanol-d₄) δ 7.88 (d, J=0.7 Hz, 2H), 7.58 (dq, J=7.9, 0.9 Hz, 1H), 7.33 (dq, J=8.2, 0.8 Hz, 1H), 7.11-7.05 (m, 2H), 7.02-6.95 (m, 2H), 3.64 (t, J=7.3 Hz, 2H), 3.05 (t, J=7.2 Hz, 2H). ¹³C NMR (126 MHz, Methanol-d₄) δ 165.7, 138.3, 136.4, 129.2, 127.7, 122.8, 122.3, 121.2, 118.4, 118.1, 112.1, 111.1, 41.2, 24.9. HRMS (ESI) calculated for [C₁₇H₁₄Br₂N₂O+H]⁺: 420.9546. Found: 420.9544. IR νₘₐₓ (cm⁻¹): 3409, 3260, 1636, 1542, 735, 694. UV-VIS λₘₐₓ: 290 nm.

N-(2-(1H-indol-3-yl)ethyl)-3,5-bis(trifluoromethyl)benzamide (1c). Using the general procedure for amide formation with tryptamine and 3,5-bis(trifluoromethyl)benzoyl chloride, 1c was obtained as a white solid in 100% yield. ¹H NMR (500 MHz, Methanol-d₄) δ 8.32 (s, 2H), 8.12 (d, J=3.3, 0.9 Hz, 1H), 7.57 (d, J=7.9, 1H), 7.35-7.30 (m, 1H), 7.12-7.03 (m, 2H), 7.00-6.93 (m, 1H), 3.70 (t, J=7.2 Hz, 2H), 3.09 (t, J=7.2 Hz, 2H). ¹³C NMR (125 MHz, Methanol-d₄) δ 165.3, 137.0, 136.8, 131.6 (q, J=33.6 Hz), 127.5, 124.5, 124.4, 122.1, 121.8, 120.9, 118.2, 117.9, 111.9, 110.9, 41.1, 24.6. HRMS (ESI) calculated for [C₁₉H₁₄F₆N₂O+H]⁺: 401.1083. Found: 401.1071. IR νₘₐₓ (cm⁻¹): 3327, 1606, 1538, 1266, 1127, 731, 699. UV-VIS λₘₐₓ: 288 nm.

N-(2-(1H-indol-3-yl)ethyl)-3,4-dichlorobenzamide (1d). Using the general procedure for amide formation with tryptamine and 3,4-dichlorobenzoyl chloride, 1d was obtained as a white-yellow solid in 90% yield. ¹H NMR (400 MHz, Methanol-d₄) δ 7.80 (t, J=1.7 Hz, 1H), 7.55 (dt, J=8.4, 1.7 Hz, 1H), 7.47 (td, J=6.9, 1.2 Hz, 2H), 7.22 (dd, J=8.2, 1.1 Hz, 1H), 7.02-6.93 (m, 2H), 6.92-6.83 (m, 1H), 3.54 (t, J=7.3 Hz, 2H), 2.95 (t, J=7.3 Hz, 2H). ¹³C NMR (101 MHz, Methanol-d₄) δ 166.3, 136.8, 135.1, 134.7, 132.2, 130.3, 129.1, 127.4, 126.6, 122.1, 120.9, 118.2, 117.9, 111.9, 110.9, 40.9, 24.7. HRMS (ESI) calculated for [C₁₇H₁₄C₁₂N₂O+H]⁺: 333.0556. Found: 333.0540. IR νₘₐₓ (cm⁻¹): 3388, 3282, 1626, 1541, 757. UV-VIS λₘₐₓ: 288 nm.

N-(2-(5-bromo-1H-indol-3-yl)ethyl)-4-butylbenzamide (6a). Using the general procedure for amide formation with 5-bromotryptamine and 4-n-butylbenzoyl chloride, 6a was obtained as a white solid in 85% yield. ¹H NMR (400 MHz, Methanol-d₄) δ 8.47 (s, 1H, amide N—H), 7.95-7.89 (m, 1H), 7.74-7.65 (m, 2H), 7.31-7.21 (m, 3H), 7.20-7.11 (m, 2H), 3.62 (t, J=7.3 Hz, 2H), 3.02 (t, J=7.3 Hz, 2H), 2.65 (t, J=7.8 Hz, 2H), 1.68-1.54 (m, 2H), 1.43-1.26 (m, 2H), 0.94 (t, J=7.3 Hz, 3H). ¹³C NMR (101 MHz, Methanol-d₄) δ 169.0, 146.7, 135.3, 131.8, 129.4, 129.3, 128.2, 123.7, 123.6, 120.7, 112.5, 112.0, 111.5, 40.8, 35.2, 33.3, 24.7, 22.0, 12.9. HRMS (ESI) calculated for [C₂₁H₂₃BrN₂O+H]⁺: 399.1067. Found: 399.1047. IR νₘₐₓ (cm⁻¹): 3250, 2917, 1603, 1560, 1306, 674, 625. UV-VIS λₘₐₓ: 290 nm.

3,5-dibromo-N-(2-(5-bromo-1H-indol-3-yl)ethyl)benzamide (6b). Using the general procedure for amide formation with 5-bromotryptamine and 3,5-dibromobenzoyl chloride, 6b was obtained as a white solid in 89% yield. ¹H NMR (400 MHz, Methanol-d₄) δ 7.90-7.84 (m, 3H), 7.68 (d, J=1.8 Hz, 1H), 7.25 (d, J=8.6 Hz, 1H), 7.19-7.11 (m, 2H), 3.61 (t, J=7.1 Hz, 2H), 3.01 (t, J=7.2, 2H). ¹³C NMR (101 MHz, Methanol-d₄) δ 165.6, 138.0, 136.2, 135.3, 129.4, 129.0, 123.7, 123.6, 122.7, 120.6, 112.5, 111.8, 111.5, 41.1, 24.4. HRMS (ESI) calculated for [C₁₇H₁₃Br₃N₂O+H]⁺: 498.8651. Found: 498.8660. IR νₘₐₓ (cm⁻¹): 3405, 3078, 1634, 1542, 797, 583. UV-VIS λₘₐₓ: 292 nm.

N-(2-(5-bromo-1H-indol-3-yl)ethyl)-3,5-bis(trifluoromethyl)benzamide (6c). Using the general procedure for amide formation with 5-bromotryptamine and 3,5-bis(trifluoromethyl)benzoyl chloride, 6c was obtained as a white-tan solid in 100% yield. ¹H NMR (400 MHz, Methanol-d₄) δ 8.32 (s, 2H), 8.16-8.10 (m, 1H), 7.66 (d, J=1.7 Hz, 1H), 7.28-7.22 (m, 1H), 7.18-7.10 (m, 2H), 3.67 (t, J=7.1 Hz, 2H), 3.06 (t, J=7.1 Hz, 2H). ¹³C NMR (101 MHz, Methanol-d₄) δ 165.3, 136.8, 135.3, 131.6 (q, J=33.7 Hz), 129.4, 124.5, 124.4, 123.7, 123.6, 121.8, 120.6, 112.5, 111.9, 111.5, 41.2, 24.3. HRMS (ESI) calculated for [C₁₉H₁₃BrF₆N₂O+H]⁺: 479.0115. Found: 479.0176. IR νₘₐₓ (cm⁻¹): 3301, 1644, 1274, 1124, 794, 700. UV-VIS λₘₐₓ: 292 nm.

N-(2-(5-bromo-1H-indol-3-yl)ethyl)-3,4-dichlorobenzamide (6d). Using the general procedure for amide formation with 5-bromotryptamine and 3,4-dichlorobenzoyl chloride, 6d was obtained as a white solid in 91% yield. ¹H NMR (400 MHz, Methanol-d₄) δ 7.89 (d, J=2.1 Hz, 1H), 7.72-7.63 (m, 2H), 7.59 (d, J=8.4 Hz, 1H), 7.28-7.22 (m, 1H), 7.19-7.11 (m, 2H), 3.62 (t, J=7.2 Hz, 2H), 3.02 (t, J=7.2, 2H). ¹³C NMR (101 MHz, Methanol-d₄) δ 166.3, 135.3, 135.1, 134.7, 132.3, 130.3, 129.4, 129.1, 126.6, 123.7, 123.6, 120.6, 112.5, 111.9, 111.5, 41.0, 24.5. HRMS (ESI) calculated for [C₁₇H₁₃BrCl₂N₂O+H]⁺: 410.9661. Found: 410.9660. IR νₘₐₓ (cm⁻¹): 3423, 3207, 1652, 1503, 797, 750. UV-VIS λₘₐₓ: 292 nm.

1-(2-(1H-indol-3-yl)ethyl)-3-(4-butylphenyl)urea (8a). Using the general procedure for urea formation via an isocyanate with tryptamine and 4-n-butylphenyl isocyanate, 8a was obtained as a white solid in 48% yield. ¹H NMR (700 MHz, Methanol-d₄) δ 7.60 (dt, J=7.9, 1.0 Hz, 1H), 7.36 (d, J=8.2 Hz, 1H), 7.23-7.21 (m, 2H), 7.14-7.06 (m, 4H), 7.04-7.00 (m, 1H), 3.53 (t, J=7.0 Hz, 2H), 3.00 (t, J=7.0 Hz, 2H), 2.57 (t, J=7.7 Hz, 2H), 1.64-1.55 (m, 2H), 1.42-1.34 (m, 2H), 0.96 (t, J=7.4 Hz, 3H). ¹³C NMR (175 MHz, Methanol-d₄) δ 157.2, 137.0, 136.8(d), 128.3, 127.4, 122.1, 120.9, 119.2, 118.2, 118.0, 111.9, 110.8, 40.3, 34.6, 33.7, 25.6, 21.9, 12.9. HRMS (ESI) calculated for [C₂₁H₂₅N₃O+H]⁺: 336.2070. Found: 336.2049. IR νₘₐₓ (cm⁻¹): 3371, 3269, 3055, 2922, 2444, 1619, 1592, 1506, 1244, 1232, 835, 737. UV-VIS λₘₐₓ: 290 nm.

1-(2-(1H-indol-3-yl)ethyl)-3-(3,5-dibromophenyl)urea (8b). Using the general procedure for urea formation via an aniline with tryptamine and 3,5-dibromoaniline, 8b was obtained as a tan solid in 26% yield. ¹H NMR (700 MHz, Methanol-d₄) δ 7.62-7.56 (m, 3H), 7.36 (d, J=8.1 Hz, 1H), 7.27 (t, J=1.7 Hz, 1H), 7.14-7.09 (m, 2H), 7.03 (t, J=7.5 Hz, 1H), 3.53 (t, J=7.0 Hz, 2H), 3.00 (t, J=7.0 Hz, 2H). ¹³C NMR (175 MHz, Methanol-d₄) δ 156.0, 142.6, 136.9, 127.4, 126.4, 122.4, 122.2, 121.0, 119.5, 118.3, 117.9, 111.7, 110.9, 40.2, 25.4. HRMS (ESI) calculated for [C₁₇H₁₅Br₂N₃O+H]⁺: 435.9655. Found: 435.9629. IR νₘₐₓ (cm⁻¹): 3393, 3309, 2919, 2850, 1633, 1591, 1259, 1224, 852, 823, 738, 663. UV-VIS λₘₐₓ: 288 nm.

1-(2-(1H-indol-3-yl)ethyl)-3-(3,5-bis(trifluoromethyl)phenyl)urea (8c). Using the general procedure for urea formation via an aniline with tryptamine and 3,5-bis(trifluoromethyl)aniline, 8c was obtained as a white solid in 17% yield. ¹H NMR (700 MHz, Methanol-d₄) δ 7.99 (s, 2H), 7.60 (d, J=7.9 Hz, 1H), 7.49 (s, 1H), 7.36 (d, J=8.1 Hz, 1H), 7.14-7.08 (m, 2H), 7.02 (td, J=8.0, 0.9 Hz, 1H), 3.56 (t, J=7.0 Hz, 2H), 3.02 (t, J=7.0 Hz, 2H). ¹³C NMR (175 MHz, Methanol-d₄) δ 156.0, 142.1, 137.9, 131.7 (q, J=33.0), 127.4, 124.2, 122.7, 122.4, 122.2, 121.0, 118.3, 117.9, 117.5, 113.9, 111.7, 110.9, 40.3, 25.4. HRMS (ESI) calculated for [C₁₉H₁₅F₆N₃O+H]⁺: 416.1192. Found: 416.1166. IR νₘₐₓ (cm⁻¹): 3431, 3396, 3293, 3088, 2922, 2850, 1644, 1560, 1473, 1274, 1124, 877, 679. UV-VIS λₘₐₓ: 290 nm.

1-(2-(1H-indol-3-yl)ethyl)-3-(3,4-dichlorophenyl)urea (8d). Using the general procedure for urea formation via an isocyanate with tryptamine and 3,4-dichlorophenyl isocyanate, 8d was obtained as a white-yellow solid in 78% yield. ¹H NMR (700 MHz, Methanol-d₄) δ 7.66 (d, J=2.7 Hz, 1H), 7.57 (d, J=7.9 Hz, 1H), 7.35 (d, J=8.1 Hz, 1H), 7.24 (d, J=8.8 Hz, 1H), 7.13-7.08 (m, 2H), 7.06 (s, 1H), 7.02 (td, J=7.3, 1.0 Hz, 1H), 3.51 (t, J=7.0 Hz, 2H), 2.96 (t, J=7.1 Hz, 2H). ¹³C NMR (175 MHz, Methanol-d₄) δ 156.3, 139.7, 136.8, 131.9, 130.0, 127.4, 124.3, 122.2, 121.1, 119.8, 118.4, 118.0, 117.9, 111.8, 111.0, 40.2, 25.5. HRMS (ESI) calculated for [C₁₇H₁₅Cl₂N₃O+H]⁺: 348.0665. Found: 348.0661. IR νₘₐₓ (cm⁻¹): 3413, 3296, 2924, 2853, 1625, 1577, 1550, 1529, 1450, 818, 738. UV-VIS λₘₐₓ: 288 nm.

1-(2-(1H-indol-3-yl)ethyl)-3-(4-bromo-3,5-dichlorophenyl)urea (8e). Using the general procedure for urea formation via an aniline with tryptamine and the product 4-bromo-3,5- dichloroaniline, 8e was obtained as a yellow solid in 38% yield. ¹H NMR (700 MHz, Methanol-d₄) δ 7.58 (d, J=7.9 Hz, 1H), 7.53 (s, 2H), 7.35 (d, J=8.1 Hz, 1H), 7.11 (t, J=8.0 Hz, 2H), 7.02 (t, J=7.5 Hz, 1H), 3.53 (t, J=7.0 Hz, 2H), 2.99 (t, J=7.0 Hz, 2H). ¹³C NMR (175 MHz, Methanol-d₄) δ 155.9, 140.6, 136.8, 135.6, 127.4, 122.2, 121.0, 118.3, 117.9, 117.8, 113.5, 111.7, 110.9, 40.2, 25.4. HRMS (ESI) calculated for [C₁₇H₁₄BrCl₂N₃O+H]⁺: 425.9770. Found: 425.9745. IR νₘₐₓ (cm⁻¹): 3470, 3432, 3320, 3089, 2953, 1638, 1589, 1538, 1515, 1453, 747, 739. UV-VIS λₘₐₓ: 288 nm.

1-(2-(5-bromo-1H-indol-3-yl)ethyl)-3-(4-butylphenyl)urea (9a). Using the general procedure for urea formation via an isocyanate with 5-bromotryptamine and 4-n-butylphenyl isocyanate, 9a was obtained as a white solid in 60% yield. ¹H NMR (500 MHz, Methanol-d₄) δ 7.72 (d, J=1.90, 0.50 Hz, 1H), 7.25 (d, J=8.65, 0.50 Hz, 1H), 7.19 (m, 3H), 7.13 (s, 1H), 7.05 (m, 2H), 3.47 (t, J=7.05 Hz, 2H), 2.93 (t, J=7.03 Hz, 2H), 2.54 (t, J=7.65 Hz, 2H), 1.56 (m, 2H), 1.34 (m, 2H), 0.92 (t, J=7.35 Hz, 3H). ¹³C NMR (125 MHz, Methanol-d₄) δ 157.2, 137.0, 136.8, 135.4, 129.3, 128.3, 123.8, 123.6, 120.6, 119.3, 112.5, 111.9, 111.4, 40.3, 34.6, 33.7, 25.4, 21.9, 12.9. HRMS (ESI) calculated for [C₂₁H₂₄BrN₃O+H]⁺: 414.1103. Found: 414.1117. IR νₘₐₓ (cm⁻¹): 3270, 2923, 1549, 1250, 801, 598. UV-VIS λₘₐₓ: 290 nm.

1-(2-(5-bromo-1H-indol-3-yl)ethyl)-3-(3,5-dibromophenyl)urea (9b). Using the general procedure for urea formation via an aniline with 5-bromotryptamine and 3,5-dibromoaniline, 9b was obtained as a white solid in 26% yield. ¹H NMR (400 MHz, Methanol-d₄) δ 7.71 (d, J=1.8 Hz, 1H), 7.57 (d, J=1.7 Hz, 2H), 7.29-7.23 (m, 2H), 7.21-7.12 (m, 2H), 3.47 (t, J=7.0 Hz, 2H), 2.94 (t, J=6.7 Hz, 1H). ¹³C NMR (101 MHz, DMSO-d₆) δ 155.1, 143.9, 135.4, 129.6, 125.7, 125.1, 123.9, 122.8, 121.1, 119.3, 113.9, 112.1, 111.4, 25.8. HRMS (ESI) calculated for [C₁₇H₁₄Br₃N₃O+H]⁺: 515.8759. Found: 515.8772. IR νₘₐₓ (cm⁻¹): 3406, 3288, 1631, 1574, 868, 583, 500. UV-VIS λₘₐₓ: 296 nm.

1-(3,5-bis(trifluoromethyl)phenyl)-3-(2-(5-bromo-1H-indol-3-yl)ethyl)urea (9c). Using the general procedure for urea formation via an aniline with 5-bromotryptamine and 3,5-bis(trifluoromethyl)aniline, 9c was obtained as a white solid in 30% yield. ¹H NMR (400 MHz, Methanol-d₄) δ 7.97 (s, 2H), 7.71 (t, J=1.6 Hz, 1H), 7.47 (s, 1H), 7.25 (dd, J=8.6, 1.7 Hz, 1H), 7.16 (dt, J=8.4, 1.7 Hz, 2H), 3.49 (td, J=7.0, 1.6 Hz, 2H), 2.95 (td, J=7.0, 2.1 Hz, 2H). ¹³C NMR (101 MHz, Methanol-d₄) δ 157.4, 143.4, 136.8, 133.1 (q, J=33.0 Hz), 130.7, 126.2, 125.2, 125.0, 123.5, 122.0, 115.4, 113.9, 113.1, 112.8, 41.8, 26.5. HRMS (ESI) calculated for [C₁₉H₁₄BrF₆N₃O+H]⁺: 494.0297. Found: 494.0281. IR νₘₐₓ (cm⁻¹): 3411, 1649, 1278, 1146, 888. UV-VIS λₘₐₓ: 296 nm.

1-(2-(5-bromo-1H-indol-3-yl)ethyl)-3-(3,4-dichlorophenyl)urea (9d). Using the general procedure for urea formation via an isocyanate with 5-bromotryptamine and 3,4-dichlorophenyl isocyanate, 9d was obtained as a white solid in 70% yield. ¹H NMR (500 MHz, Methanol-d₄) δ 7.61 (d, J=1.90 Hz, 1H), 7.60 (d, J=2.50 Hz, 1H), 7.24 (d, J=8.75 Hz, 1H), 7.16 (d, J=8.60 Hz, 1H), 7.07 (m, 3H), 3.37 (t, J=6.95 Hz, 2H), 2.84 (t, J=6.95 Hz, 2H). ¹³C NMR (125 MHz, DMSO-d₆) δ 155.3, 141.3, 135.4, 131.3, 130.9, 129.6, 125.0, 123.8, 122.5, 121.1, 119.1, 118.1, 113.9, 112.1, 111.4, 40.2, 25.9. HRMS (ESI) calculated for [C₁₇H₁₄BrCl₂N₃O+H]⁺: 425.9770. Found: 425.9738. IR νₘₐₓ (cm⁻¹): 3316, 1622, 1567, 817, 787, 587. UV-VIS λₘₐₓ: 288 nm.

1-(2-(5-bromo-1H-indol-3-yl)ethyl)-3-(4-bromo-3,5-dichlorophenyl)urea (9e). Using the general procedure for urea formation via an aniline with 5-bromotryptamine and the product 4-bromo-3,5-dichloroaniline, 9e was obtained as a white solid in 38% yield. ¹H NMR (500 MHz, Acetone-d₆) δ 10.26 (s, 1H), 8.33 (s, 1H), 7.78-7.71 (m, 3H), 7.35 (dd, J=8.6, 1.5 Hz, 1H), 7.25 (s, 1H), 7.19 (dt, J=8.7, 1.8 Hz, 1H), 6.04 (s, 1H), 3.55-3.48 (m, 2H), 2.95 (t, J=7.1 Hz, 2H). ¹³C NMR (101 MHz, DMSO-d₆) δ 155.1, 142.0, 135.4, 135.3, 125.1, 123.9, 121.1, 119.0, 117.9, 113.9, 112.6, 112.1, 111.4, 25.8. HRMS (ESI) calculated for [C₁₇H₁₃Br₂Cl₂N₃O+H]⁺: 505.8782. Found: 505.8760. IR νₘₐₓ (cm⁻¹): 3449, 3299, 2907, 1627, 1450, 787, 756, 444. UV-VIS λₘₐₓ: 290 nm.

1-((1H-indol-3-yl)methyl)-3-(4-butylphenyl)urea (11a). Using the general procedure for urea formation via an isocyanate with (1H-indol-3-yl)methanamine and 4-n-butylphenyl isocyanate, 11a was obtained as a red solid in 10% yield. ¹H NMR (700 MHz, Methanol-d₄) δ 7.64 (d, J=7.9 Hz, 1H), 7.38 (d, J=8.2 Hz, 1H), 7.27 (d, J=8.7 Hz, 2H), 7.24 (s, 1H), 7.13 (t, J=7.6 Hz, 1H), 7.09 (d, J=8.3 Hz, 2H), 7.05 (t, J=7.5 Hz, 1H), 4.57 (s, 2H), 2.57 (t, J=7.7 Hz, 2H), 1.62-1.55 (m, 2H), 1.41-1.34 (m, 2H), 0.95 (t, J=7.4 Hz, 3H). ¹³C NMR (175 MHz, Methanol-d₄) δ 157.0, 137.1, 136.9, 136.8, 128.3, 126.6, 122.9, 121.2, 119.1, 118.6, 118.1, 112.4, 110.9, 35.1, 34.6, 33.7, 21.9, 12.9. HRMS (ESI) calculated for [C₂₀H₂₃N₃O+H]⁺: 322.1914. Found: 322.1895. IR νₘₐₓ (cm⁻¹): 3281, 3052, 2954, 2922, 2856, 1589, 1571, 1513, 1427, 1229, 829, 736. UV-VIS λₘₐₓ: 288 nm.

1-((1H-indol-3-yl)methyl)-3-(3,5-dibromophenyl)urea (11b). Using the general procedure for urea formation via an aniline with (1H-indol-3-yl)methanamine and 3,5-dibromoaniline, 11b was obtained as a brown solid in 12% yield. ¹H NMR (700 MHz, Methanol-d₄) δ 7.65-7.60 (m, 3H), 7.37 (d, J=8.1 Hz, 1H), 7.27 (t, J=1.6 Hz, 1H), 7.24 (s, 1H), 7.13 (t, J=7.5 Hz, 1H), 7.05 (t, J=7.4 Hz, 1H), 4.56 (s, 2H). ¹³C NMR (175 MHz, Methanol-d₄) δ 155.8, 142.6, 136.9, 126.5, 126.4, 123.0, 122.4, 121.3, 119.4, 118.7, 118.0, 112.0, 111.0, 35.0. HRMS (ESI) calculated for [C₁₆H₁₃Br₂N₃O+H]⁺: 421.9498. Found: 421.9466. IR νₘₐₓ (cm⁻¹): 3301, 2920, 2852, 1535, 1415, 735. UV-VIS λₘₐₓ: 288 nm.

1-((1H-indol-3-yl)methyl)-3-(3,5-bis(trifluoromethyl)phenyl)urea (11c). Using the general procedure for urea formation via an aniline with (1H-indol-3-yl)methanamine and 3,5-bis(trifluoromethyl)aniline, 11c was obtained as a tan solid in 35% yield. ¹H NMR (700 MHz, Methanol-d₄) δ 8.03 (s, 2H), 7.64 (d, J=7.9 Hz, 1H), 7.49 (s, 1H), 7.37 (d, J=8.2 Hz, 1H), 7.24 (s, 1H), 7.13 (t, J=7.6 Hz, 1H), 7.05 (t, J=7.5 Hz, 1H), 4.59 (s, 2H). ¹³C NMR (175 MHz, Methanol-d₄) δ 155.8, 142.0, 136.9, 131.7 (q, J=33.0 Hz), 126.5, 123.1, 121.3, 118.7, 118.0, 117.5, 114.0, 111.9, 111.0, 35.1. HRMS (ESI) calculated for [C₁₈H₁₃F₆N₃O+H]⁺: 402.1036. Found: 402.1012. IR νₘₐₓ (cm⁻¹): 3400, 3377, 3250, 2922, 1672, 1543, 1523, 878, 752, 699,679. UV-VIS λₘₐₓ: 288 nm.

1((1H-indol-3-yl)methyl)-3-(3,4-dichlorophenyl)urea (11d). Using the general procedure for urea formation via an isocyanate with (1H-indol-3-yl)methanamine and 3,4-dichlorophenyl isocyanate, 11d was obtained as a white-yellow solid in 60% yield. ¹H NMR (700 MHz, Methanol-d₄) δ 7.73 (d, J=2.1 Hz, 1H), 7.62 (d, J=7.9 Hz, 1H), 7.37 (d, J=8.1 Hz, 1H), 7.33 (d, J=8.7 Hz, 1H), 7.24-7.18 (m, 2H), 7.13 (t, J=7.5 Hz, 1H), 7.04 (t, J=7.5 Hz, 1H), 4.56 (s, 2H). ¹³C NMR (175 MHz, Methanol-d₄) δ 156.1, 139.9, 136.9, 131.9, 130.0, 126.5, 124.3, 123.0, 121.3, 119.7, 118.7, 118.0, 117.9, 112.1, 111.0, 35.1. HRMS (ESI) calculated for [C₁₆H₁₃Cl₂N₃O+H]⁺: 334.0508. Found: 334.0484. IR νₘₐₓ (cm⁻¹): 3309, 1577, 1472, 1373, 1227, 738. UV-VIS λₘₐₓ: 288 nm.

1((1H-indol-3-yl)methyl)-3-(4-bromo-3,5-dichlorophenyl)urea (11e). Using the general procedure for urea formation via an aniline with (1H-indol-3-yl)methanamine and the product 4-bromo-3,5-dichloroaniline, 11e was obtained as a yellow solid in 32% yield. ¹H NMR (600 MHz, Methanol-d₄) δ 7.62-7.57 (m, 3H), 7.35 (dd, J=8.2, 1.5 Hz, 1H), 7.22 (s, 1H), 7.15-7.08 (m, 1H), 7.08-6.99 (m, 1H), 4.54 (s, 2H). ¹³C NMR (175 MHz, Methanol-d₄) δ 155.7, 140.7, 136.9, 135.6, 126.5, 123.1, 121.3, 118.7, 118.0, 117.8, 113.4, 112.0, 111.0, 35.1. HRMS (ESI) calculated for [C₁₆H₁₂BrCl₂N₃O+Na]⁺: 413.9520. Found: 413.9533. IR νₘₐₓ (cm⁻¹): 3298, 1566, 1428, 1359, 1228, 736. UV-VIS λₘₐₓ: 288 nm.

1-(4-butylphenyl)-3-(1H-indol-5-yl)urea (12a). Using the general procedure for urea formation via an isocyanate with 5-aminoindole and 4-n-butylphenyl isocyanate, 12a was obtained as a light brown solid in 21% yield. ¹H NMR (700 MHz, Methanol-d₄) δ 7.62 (s, 1H), 7.36-7.31 (m, 3H), 7.25-7.22 (m, 1H), 7.13-7.08 (m, 3H), 6.42 (d, J=3.0 Hz, 1H), 2.58 (t, J=7.7 Hz, 2H), 1.63-1.56 (m, 2H), 1.42-1.33 (m, 2H), 0.96 (t, J=7.4 Hz, 3H). ¹³C NMR (175 MHz, Methanol-d₄) δ 155.4, 137.0, 136.9, 133.5, 130.2, 128.3, 128.2, 125.0, 119.3, 116.3, 112.5, 110.8, 100.9, 34.6, 33.7, 21.9, 12.9. HRMS (ESI) calculated for [C₁₉H₂₁N₃O+H]⁺: 308.1757. Found: 308.1743. IR νₘₐₓ (cm⁻¹): 3421, 3297, 2955,1626, 1541, 1512, 1224, 723. UV-VIS λₘₐₓ: 288 nm.

1-(3,5-dibromophenyl)-3-(1H-indol-5-yl)urea (12b). Using the general procedure for urea formation via an aniline with 5-aminoindole and 3,5-dibromoaniline, 12b was obtained as a tan solid in 8% yield. ¹H NMR (700 MHz, Methanol-d₄) δ 7.70 (d, J=1.6 Hz, 2H), 7.62 (d, J=2.0 Hz, 1H), 7.36 (d, J=8.6 Hz, 1H), 7.33 (t, J=1.7 Hz, 1H), 7.25 (d, J=3.1 Hz, 1H), 7.11 (dd, J=8.6, 2.0 Hz, 1H), 6.43 (d, J=3.1 Hz, 1H). ¹³C NMR (175 MHz, Methanol-d₄) δ 154.3, 142.4, 133.7, 129.7, 128.2, 126.7, 125.1, 122.4, 119.8, 119.4, 118.1, 116.3, 112.6, 110.9, 100.9. HRMS (ESI) calculated for [C₁₅H₁₁Br₂N₃O+H]⁺: 407.9342. Found: 407.9316. IR νₘₐₓ (cm⁻¹): 3408, 3298, 1624, 1573, 1458, 1409, 1225, 840, 755, 730. UV-VIS λₘₐₓ: 298 nm.

1-(3,5-bis(trifluoromethyl)phenyl)-3-(1H-indol-5-yl)urea (12c). Using the general procedure for urea formation via an aniline with 5-aminoindole and 3,5-bis(trifluoromethyl)aniline, 12c was obtained as a light green solid in 67% yield. ¹H NMR (700 MHz, Methanol-d₄) δ 8.06 (s, 2H), 7.63 (d, J=2.1 Hz, 1H), 7.51 (s, 1H), 7.34 (d, J=8.6 Hz, 1H), 7.21 (d, J=3.1 Hz, 1H), 7.11 (dd, J=8.6, 2.1 Hz, 1H), 6.41 (d, J=3.1 Hz, 1H). ¹³C NMR (175 MHz, Methanol-d₄) δ 154.4, 141.8, 133.7, 131.7 (q, J=33.0 Hz), 129.4, 128.2, 125.2, 124.2, 122.7, 117.9, 116.5, 114.3, 113.0, 110.9, 101.0. HRMS (ESI) calculated for [C₁₇H₁₁F₆N₃O+H]⁺: 388.0879. Found: 388.0873. IR νₘₐₓ (cm⁻¹): 3411, 3311, 2922, 1636, 1545, 1277, 1176, 1121, 892, 728, 700, 683. UV-VIS λₘₐₓ: 292 nm.

1-(3,4-dichlorophenyl)-3-(1H-indol-5-yl)urea (12d). Using the general procedure for urea formation via an isocyanate with 5-aminoindole and 3,4-dichlorophenyl isocyanate, 12d was obtained as a white solid in 47% yield. ¹H NMR (700 MHz, Methanol-d₄) δ 7.81 (d, J=2.5 Hz, 1H), 7.62 (s, 1H), 7.40 (d, J=8.7 Hz, 1H), 7.36 (d, J=8.6 Hz, 1H), 7.30 (dd, J=8.8, 2.5 Hz, 1H), 7.25 (d, J=3.1 Hz, 1H), 7.10 (dd, J=8.6, 2.0 Hz, 1H), 6.43 (d, J=3.0 Hz, 1H). ¹³C NMR (175 MHz, Methanol-d₄) δ 154.5, 139.7, 133.6, 131.9, 130.0, 129.8, 128.2, 125.1, 124.5, 119.9, 118.1, 116.3, 112.6, 110.8, 100.9. HRMS (ESI) calculated for [C₁₅H₁₁Cl₂N₃O+H]⁺: 320.0352. Found: 320.0341. IR νₘₐₓ (cm⁻¹): 3450, 3283, 2919, 2847, 1622, 1574, 1555, 1472, 758, 724. UV-VIS λₘₐₓ: 296 nm.

1-(4-bromo-3,5-dichlorophenyl)-3-(1H-indol-5-yl)urea (12e). Using the general procedure for urea formation via an aniline with 5-aminoindole and the product 4-bromo-3,5-dichloroaniline, 12e was obtained as a white solid in 60% yield. ¹H NMR (400 MHz, DMSO-d₆) δ 11.00 (s, 1H), 9.03 (s, 1H), 8.68 (s, 1H), 7.75 (s, 2H), 7.67 (t, J=2.1 Hz, 1H), 7.31 (dd, J=5.9, 2.9 Hz, 2H), 7.08 (dd, J=8.6, 2.1 Hz, 1H), 6.36 (d, J=2.9 Hz, 1H). ¹³C NMR (101 MHz, DMSO-d₆) δ 153.0, 141.6, 135.4, 133.0, 131.1, 128.1, 126.4, 118.3, 115.6, 113.1, 111.8, 111.2, 101.4. HRMS (ESI) calculated for [C₁₅H₁₀BrCl₂N₃O+H]⁺: 397.9457. Found: 397.9448. IR νₘₐₓ(cm⁻¹): 3388, 3201, 1626, 1543, 756, 720. UV-VIS λₘₐₓ: 290 nm.

1-(4-butylphenyl)-3-(1H-indol-6-yl)urea (13a). Using the general procedure for urea formation via an isocyanate with 6-aminoindole and 4-n-butylphenyl isocyanate, 13a was obtained as a white solid in 41% yield. ¹H NMR (700 MHz, Methanol-d₄) δ 7.69-7.67 (m, 1H), 7.47 (d, J=8.4 Hz, 1H), 7.34 (d, J=8.4 Hz, 2H), 7.18 (d, J=3.1 Hz, 1H), 7.15-7.06 (m, 2H), 6.90 (dd, J=8.4, 1.9 Hz, 1H), 6.40 (dd, J=3.2, 0.9 Hz, 1H), 2.59 (t, J=7.7 Hz, 2H), 1.64-1.57 (m, 2H), 1.42-1.34 (m, 2H), 0.97 (t, J=7.4 Hz, 3H). ¹³C NMR (175 MHz, Methanol-d₄) δ 154.8, 137.1, 136.8, 136.5, 132.9, 128.3, 124.5, 124.0, 119.8, 119.3, 113.0, 102.7, 100.8, 34.6, 33.7, 21.9, 12.9. HRMS (ESI) calculated for [C₁₉H₂₁N₃O+H]⁺: 308.1757. Found: 308.1739. IR νₘₐₓ (cm⁻¹): 3308, 3034, 2953, 2924, 2870, 2854, 1625, 1606, 1589, 1541, 1513, 1451, 1412, 1226. UV-VIS λₘₐₓ: 298 nm.

1-(3,5-dibromophenyl)-3-(1H-indol-6-yl)urea (13b). Using the general procedure for urea formation via an aniline with 6-aminoindole and 3,5-dibromoaniline, 13b was obtained as a white solid in 6% yield. ¹H NMR (700 MHz, Methanol-d₄) δ 7.71 (s, 2H), 7.69 (s, 1H), 7.49 (d, J=8.4 Hz, 1H), 7.34 (d, J=1.7 Hz, 1H), 7.20 (d, J=2.9 Hz, 1H), 6.90 (dd, J=8.4, 1.8 Hz, 1H), 6.41 (d, J=3.1 Hz, 1H). ¹³C NMR (175 MHz, Methanol-d₄) δ 153.7, 142.4, 136.4, 132.3, 126.8, 124.8, 124.2, 122.4, 119.9, 119.8, 113.0, 102.9, 100.8. HRMS (ESI) calculated for [C₁₅H₁₁Br₂N₃O+H]⁺: 409.9322. Found: 409.9292. IR νₘₐₓ (cm⁻¹): 3301, 3055, 2924, 1608, 1569, 1541, 850, 722. UV-VIS λₘₐₓ: 296 nm.

1-(3,5-bis(trifluoromethyl)phenyl)-3-(1H-indol-6-yl)urea (13c). Using the general procedure for urea formation via an aniline with 6-aminoindole and 3,5-bis(trifluoromethyl)aniline, 13c was obtained as a white solid in 19% yield. ¹H NMR (700 MHz, Methanol-d₄) δ 8.09 (s, 2H), 7.71 (s, 1H), 7.53 (s, 1H), 7.49 (d, J=8.4 Hz, 1H), 7.18 (d, J=3.2 Hz, 1H), 6.92 (dd, J=8.4, 1.9 Hz, 1H), 6.41 (d, J=3.1 Hz, 1H). ¹³C NMR (175 MHz, Methanol-d₄) δ 153.8, 141.8, 136.4, 132.1, 131.8 (q, J=33.1 Hz), 125.8, 124.9, 124.2, 122.7, 121.1, 119.9, 117.9, 114.3, 113.1, 103.1, 100.9. HRMS (ESI) calculated for [C₁₇H₁₁F₆N₃O+H]⁺: 388.0879. Found: 388.0874. IR νₘₐₓ (cm⁻¹): 3284, 2953, 2922, 1608, 1547, 1275, 894, 726, 703 682. UV-VIS λₘₐₓ: 298 nm.

1-(3,4-dichlorophenyl)-3-(1H-indol-6-yl)urea (13d). Using the general procedure for urea formation via an isocyanate with 6-aminoindole and 3,4-dichlorophenyl isocyanate, 13d was obtained as a white-yellow solid in 10% yield. ¹H NMR (700 MHz, Methanol-d₄) δ 7.83 (d, J=2.4 Hz, 1H), 7.69 (s, 1H), 7.49 (d, J=8.4 Hz, 1H), 7.42 (d, J=8.8 Hz, 1H), 7.31 (dd, J=8.9, 2.4 Hz, 1H), 7.20 (d, J=3.1 Hz, 1H), 6.90 (d, J=8.3 Hz, 1H), 6.41 (d, J=3.1 Hz, 1H). ¹³C NMR (175 MHz, Methanol-d₄) δ 154.0, 139.6, 136.4, 132.4, 131.9, 130.1, 124.7, 124.5, 124.1, 119.9, 119.9, 118.1, 113.0, 102.8, 100.8. HRMS (ESI) calculated for [C₁₅H₁₁Cl₂N₃O+H]⁺: 320.0352. Found: 320.0340. IR νₘₐₓ (cm⁻¹): 3298, 2952, 2922, 1613, 1578, 1545, 1472, 1228, 1125, 810, 767, 725. UV-VIS λₘₐₓ: 298 nm.

1-(4-bromo-3,5-dichlorophenyl)-3-(1H-indol-6-yl)urea (13e). Using the general procedure for urea formation via an aniline with 6-aminoindole and the product 4-bromo-3,5-dichloroaniline, 13e was obtained as a light brown solid in 65% yield. ¹H NMR (400 MHz, DMSO-d₆) δ 10.97 (s, 1H), 9.06 (s, 1H), 8.83 (s, 1H), 7.75 (s, 3H), 7.43 (d, J=8.4 Hz, 1H), 7.25 (t, J=2.7 Hz, 1H), 6.89 (dd, J=8.4, 1.9 Hz, 1H), 6.38-6.32 (m, 1H). ¹³C NMR (101 MHz, DMSO) δ 152.8, 141.5, 136.6, 135.4, 133.4, 125.2, 124.0, 120.4, 118.3, 112.7, 102.2, 101.4. HRMS (ESI) calculated for [C₁₅H₁₀BrCl₂N₃O+H]⁺: 397.9457. Found: 397.9444. IR νₘₐₓ (cm⁻¹): 3299, 1570, 1545, 807, 725. UV-VIS λₘₐₓ: 294 nm.

### 4. Pharmaceutical Compositions and Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire®). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension, or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

The disclosed compounds may be incorporated into pharmaceutical compositions suitable for administration to a subject (such as a patient, which may be a human or non-human).

The pharmaceutical compositions may include a therapeutically effective amount of an agent.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp.587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active [e.g., compound of formula (I)] and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound [e.g., a compound of formula (I)], and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound [e.g., a compound of formula (I)], and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

### 5. Biological Activity

### Summary

The results described below show novel tryptamine derivatives enhance colistin efficacy against a range of MDR Gram-negative bacteria. Seven compounds were identified that, at 30 μM or less, increased colistin efficacy, reducing the MIC below the susceptible breakpoints defined by Clinical and Laboratory Standards Institute (2 μg/mL for the bacteria tested). Three of the adjuvants disclosed in this study display high colistin potentiation activity at 20 μM against all colistin resistant strains tested.

Compound 9e, at 5 μM, elicited a 1024-fold reduction of the colistin MIC against a chromosomally resistant K. pneumoniae strain, reducing the MIC from 512 to 0.5 μg/mL. 9e resensitized all three mcr-1 containing bacterial strains to colistin at a concentration of 5 μM and resensitized three out of four chromosomally resistant strains at a concentration of 7.5 μM, which makes 9e the most active adjuvant reported for reversal of colistin resistance. Further studies showed that compound 9e was generally non-toxic to red blood cells and the 4T1 cell line. Finally, an in vivo G. mellonella model showed 47% survival of inoculated wax worms after 4 days when given a combination treatment of colistin and compound 9e. This is a significant increase in survival when compared to day 4 of monotherapy of colistin (7% survival), compound 9e alone (0% survival), and 100 mg/kg of tigecycline (20% survival).

### General Methods

Broth microdilution method for MIC determination. Day cultures (6 h) of each bacterial strain in cation adjusted Mueller Hinton broth (CAMHB, Becton Dickinson) were subcultured to 5×10⁵ CFU/mL in CAMHB. Aliquots (1 mL) were placed in culture tubes and compound was added from 10 mM stock samples in DMSO, such that compound concentration equaled highest concentration tested (200 μM). Samples were then aliquoted (200 μL) into the first wells of a 96-well plate, with all remaining wells being filled with 100 μL of initial bacterial subculture. Row one wells were mixed five times, before 100 μL was transferred to row two. Row two was then mixed five times, and 100 μL was transferred to row three. This process was repeated until the final row had been mixed; this served to serially dilute the compound. Plates were then covered with GLAD Press n' Seal and incubated under stationary conditions at 37° C. for 16 h. MIC values were then recorded as the lowest concentration at which no bacterial growth was observed.

Broth microdilution method for antibiotic potentiation. Day cultures (6 h) of bacteria in CAMHB were subcultured to 5×10⁵ CFU/mL in CAMHB. Aliquots (4 mL) were placed in culture tubes and dosed with compound from 10 mM stock samples to give the desired concentration of the compound to be tested against the particular bacterial strain; this ensured non-toxic DMSO concentrations ≤0.3% in each well. 1 mL of the resulting solution was placed in a separate culture tube and dosed with antibiotic at the highest concentration to be tested. Bacteria treated with antibiotic alone served as the control. Row one of a 96-well plate was filled with 200 μL of the antibiotic/2-AI solution, and rows 2-12 were filled with 100 μL each of the remaining 4 mL of bacterial subculture containing adjuvant at the desired concentration, except for the control lane which contained only bacterial subculture. Row one was then mixed five times, and 100 μL was transferred to row two, which was then mixed five times before being transferred to row three. This process was repeated until all rows had been mixed, except for row twelve, which would have only 2-AI, to serve as a control. The 96-well plate was then covered in Glad Press n' Seal and incubated under stationary conditions at 37° C. for 16 h. MIC values were determined as the lowest concentration at which no bacterial growth was observed, and fold reductions were determined by comparison to control lane.

Broth microdilution method for antibiotic potentiation with mcr-1 harboring strains. Day cultures (6 h) of mcr-1 harboring strains in CAMHB dosed with 30 μg/mL gentamicin sulfate were subcultured to 5×10⁵ CFU/mL in CAMHB. Then the procedure for ‘broth microdilution method for antibiotic potentiation’ was followed, starting with making 4 mL aliquots.

Broth microdilution method for antibiotic potentiation with detergent. Day cultures (6 h) of bacteria in CAMHB were subcultured to 5×10⁵ CFU/mL in CAMHB and then Tween™ 80 Surfact-Amps™ Detergent Solution (Fisher Scientific, U.S.) was added to make a 0.01% containing bacterial solution. Then procedure for ‘broth microdilution method for antibiotic potentiation’ was followed starting with making 4 mL aliquots from Tween™ 80 bacterial solution.

Hemolysis assay. Hemolysis assays were performed on mechanically defibrinated sheep blood (Hemostat Labs: DSB30). Defibrinated blood (1.5 mL) was placed into a microcentrifuge tube and centrifuged for 10 min at 10,000 rpm. The supernatant was then removed, and the cells were resuspended in 1 mL of phosphate-buffered saline (PBS). The suspension was centrifuged, the supernatant was removed, and cells were resuspended two additional times. The final cell suspension was then diluted 10-fold. Test compound solutions were made in PBS in small culture tubes and then added to aliquots of the 10-fold suspension dilution of blood. PBS was used as a negative control and a zero hemolysis marker. Triton X (a 1% sample) was used as a positive control serving as the 100% lysis marker. Samples were then placed in an incubator at 37° C. while being shaken at 200 rpm for one hour. After one hour, the samples were transferred to microcentrifuge tubes and centrifuged for 10 min at 10,000 rpm. The resulting supernatant was diluted by a factor of 40 in distilled water. The absorbance of the supernatant was then measured with a UV spectrometer at a 540 nm wavelength.

MTT Cell Viability Assay. 4T1 cells (ATCC Manassas, Va.) were plated at a density of 1×10⁴ cells/well in 96-well plates. (Roswell Park Memorial Institute Media 1640, Gibco, Gaithersburg, Md.) supplemented with 10% Fetal Bovine Serum (Gibco), 2 mM GlutaMAX (Gibco) and 50 μM 2-mercaptoethanol (Sigma Aldrich St. Louis, Mo.) and incubated at 37° C. under a 5% CO₂ atmosphere in the dark for 18 h. Cell cultures were treated with serial dilutions of compounds in the presence or absence of 1 μg/mL colistin (3 replicates per condition) and incubated for an additional 18 hours. The following control conditions were used: media only (blank), 1% Triton X100 (0% cell viability), 0.5% DMSO (100% cell viability). Each condition was then treated with 10% volume of a 5 mg/mL solution of 3-(4,5-dimethylthiazol-2-yl)-2,5-diphenyltetrazolium bromide (MTT) (Sigma Aldrich) in sterile filtered 1× phosphate buffered saline (PBS) and incubated for 2 h. at 37° C. in 5% CO₂, after which the media was aspirated and the resulting formazan crystals were resuspended in 100 μL acidified (4 mM HCl) isopropanol. The 96-well plate was then read at 540 nm on a FLUOstar Optima (BMG Labtech Cary, N.C.) microplate reader. Cell viability was calculated as a percentage using the two previously mentioned controls.

Galleria mellonella Assay. G. mellonella larvae (Speedy Worm, Alexandria, Minn.) were used within 10 days of shipment from the vendor. After reception of worms, larvae were kept in the dark at room temperature for at least 24 h. before infection. Larvae weighing between 200 to 300 mg were used in the survival assay. Using a 10 μL glass syringe (Hamilton, Reno, Nev.) fitted with a 30 G needle (Exel International, St. Petersburg, Fla.), a 5 μL solution of the desired compound(s) and concentration(s) were injected into the last left proleg. Colistin was dosed at 50 mg/kg dissolved in DI water, 2-AIs at 50 mg/kg dissolved in DMSO, tigecycline at 100 mg/kg dissolved in DMSO, and DMSO was injected for a negative control. For bacterial injections, 1 mL from an overnight culture of A. baumannii 4106 in Miller LB broth (Fisher Scientific U.S.) was subcultured into 9 mL Miller LB broth and incubated for an additional 3 h before use. The 10 mL solution was centrifuged and washed three times with phosphate buffer solution (PBS, Fisher Scientific U.S.) before diluting to 6×10⁵ CFUs in PBS. Then, 2.5 h. after the first injection, a second 5 μL injection containing 6×10⁵ CFUs of A. baumannii 4106 was injected into the second to last left proleg. Injected worms were left at room temperature in the dark while being assessed at 24 h intervals over 4 days. Larvae were considered dead if they did not respond to physical stimuli. Experiment was repeated 3 times using 10 larvae per experimental group. No ethics approval was needed for G. mellonella.

### Example 1

Compound 1a was evaluated for its ability to potentiate colistin activity against three representative Gram-negative strains that contain the mcr-1 plasmid as well as their parent strains (Table 1).¹⁹ This compound showed modest activity, and at 50 μM

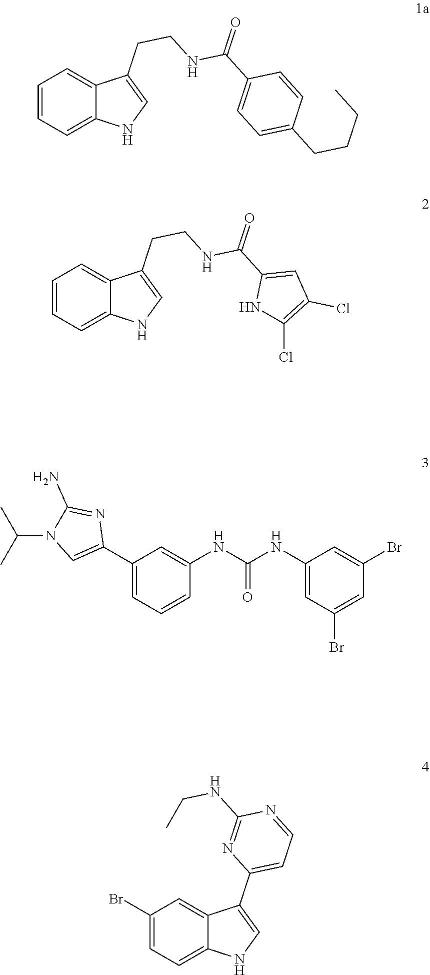 [SMILES: CCCCc1ccc(C(=O)NCCc2c[nH]c3ccccc23)cc1 ; O=C(NCCc1c[nH]c2ccccc12)c1cc(Cl)c(Cl)[nH]1 ; CC(C)n1cc(-c2cccc(NC(=O)Nc3cc(Br)cc(Br)c3)c2)nc1N ; CCNc1nccc(-c2c[nH]c3ccc(Br)cc23)n1]

reduced the colistin MICs between two and 16-fold, with the greatest activity being a 16-fold MIC reduction observed against the parent E. coli strain. In a parallel SAR study on compound 2, replacement of the halogenated pyrrole with diverse halogenated heterocycles abrogated colistin potentiation activity against several of the same bacterial strains. In contrast, compound 1a exhibited activity against these six Gram-negative strains.

TABLE 1
Potentiation of colistin (MICs μg/mL) with pilot
and brominated library adjuvants tested at 50 μM

| | E. coli | | K. pneumoniae | | A. baumannii | |
|---|---|---|---|---|---|---|
| | YD626 | | 2210291 | | 17978 | |
| Compound | Parent | *mcr-1 | Parent | *mcr-1 | Parent | *mcr-1 |
| * | 1 | 8 | 1 | 16 | 2 | 16 |
| 1a | 0.0625 | 4 | 0.125 | 8 | 0.25 | 4 |
| 1b | 0.25 | 4 | 0.5 | 4 | 1 | 4 |
| 1c | 0.0625 | 4 | 0.25 | 16 | 0.25 | 8 |
| 1d | 0.5 | 2 | 0.5 | 2 | 1 | 2 |
| 6a | 0.125 | 4 | 0.25 | 4 | 0.5 | 4 |
| 6b | 0.5 | 4 | 0.5 | 4 | 1 | 4 |
| 6c | 0.0625 | 4 | 0.25 | 8 | 0.5 | 8 |
| 6d | 0.25 | 2 | 0.5 | 1 | 0.5 | 1 |

Next, substitution of the alkyl tail and bromination of the indole ring was investigated. Seven additional compounds 1b-d and 6a-d were synthesized (Scheme 1) and assayed for activity.

The MIC of each adjuvant was measured against three engineered bacterial strains harboring the mcr-1 gene, along with the corresponding parent strains. All adjuvants were essentially non-toxic themselves, exhibiting MICs >200 μM. Each non-brominated tryptamine derivative (1b-d) was screened at 50 μM (25% of their MIC so that potential toxic effects of the adjuvants towards the bacteria would be minimal) and observed that every adjuvant showed at least modest activity against all strains tested (Table 1). Compound 1d displayed the greatest activity across all mcr-1 containing strains, lowering the colistin MIC to 2 μg/mL in all cases (between 4-8-fold reductions), which is the susceptibility breakpoint (2 μg/mL), and demonstrated that re- placement of the alkyl group is tolerated. The brominated indolic derivatives, compounds 6a-d, exhibited similar activity to their non-brominated analogues (potentiation was within two-fold against all six strains) (Table 1), with most exhibiting two-fold reductions in activity. However, compound 6d did exhibit a two-fold increase in activity against two of the three mcr-1 containing strains when compared to its non-brominated counterpart 1d.

### Example 2

Urea derivatives 8a-e and 9a-e were synthesized and assayed for adjuvant activity. As observed with other tryptamine derivatives, compounds 8a-e and 9a-e were non-toxic to bacteria and displayed standalone MICs >200 μM against the six bacterial strains. When tested at 50 μM in combination with colistin, most compounds showed increased colistin potentiation when compared to their amide counterparts (Table 2). Compounds 8b, 8c, 8d, 8e, 9d, and 9e resensitized all three mcr-1 strains towards colistin, dropping the MIC to ≤2 μg/mL, demonstrating that the urea linkage generally is more active for colistin potentiation than the amide.

TABLE 2
Potentiation of colistin (MICs μg/mL) with urea-
containing adjuvants tested at 50 μM

| | E. coli | | K. pneumoniae | | A. baumannii | |
|---|---|---|---|---|---|---|
| | YD626 | | 2210291 | | 17978 | |
| Compound | Parent | *mcr-1 | Parent | *mcr-1 | Parent | *mcr-1 |
| * | 1 | 8 | 1 | 16 | 2 | 16 |
| 8a | 1 | 8 | 1 | 16 | 2 | 16 |
| 8b | 0.25 | 2 | 0.5 | 1 | 0.5 | 1 |
| 8c | 0.0313 | 1 | 0.25 | 1 | 0.125 | 0.5 |
| 8d | 0.125 | 1 | 0.25 | 0.5 | 0.5 | 0.5 |
| 8e | 0.125 | 1 | 0.5 | 1 | 0.5 | 1 |
| 9a | 1 | 8 | 0.5 | 16 | 2 | 16 |
| 9b | 0.25 | 2 | 0.5 | 2 | 0.5 | 2 |
| 9c | 0.0313 | 4 | 0.5 | 2 | 0.25 | 2 |
| 9d | 0.25 | 0.5 | 0.5 | 0.5 | 0.5 | 1 |
| 9e | 0.25 | 1 | 0.5 | 0.5 | 0.5 | 0.5 |

### Example 3

Since the replacement of the amide with a urea moiety provided analogs with increased colistin potentiation activity, the effect of linker length between the indole and urea and the position at which the urea was attached to the indole ring was explored. Three additional sets of compounds were synthesized following the routes outlined in Schemes 2-4, but with the urea placed either one methylene group closer to the indole ring (11a-e) or directly attached to the indole ring, at either the 5 or 6 position (12/13a-e). As with all previously synthesized analogs, adjuvant standalone MICs were >200 μM, and all were therefore tested at 50 μM in combination with colistin. With the exception of compound 11c, all compound 11 derivatives exhibited a decrease in activity. Compound 11c rivaled its tryptamine derivative 8c in terms of activity. As for derivatives 12a-e and 13a-e, direct attachment of the urea to the indole ring at the 5 or 6 position abrogated activity, showing ≤2-fold reduction in the MIC of colistin (Table 3).

TABLE 3
Potentiation of colistin (MICs μg/mL) with second-generation
urea-containing adjuvants tested at 50 μM

| | E. coli | | K. pneumoniae | | A. baumannii | |
|---|---|---|---|---|---|---|
| Com- | YD626 | | 2210291 | | 17978 | |
| pound | Parent | *mcr-1 | Parent | *mcr-1 | Parent | *mcr-1 |
| * | 1 | 8 | 1 | 16 | 2 | 16 |
| 11a | 1 | 8 | 0.5 | 16 | 2 | 16 |
| 11b | 0.25 | 4 | 0.5 | 16 | 1 | 8 |
| 11c | 0.0313 | 1 | 0.125 | 1 | 0.125 | 1 |
| 11d | 0.5 | 4 | 0.5 | 16 | 2 | 16 |
| 11e | 0.25 | 8 | 0.5 | 8 | 1 | 8 |
| 12a | 0.5 | 8 | 0.5 | 16 | 1 | 16 |
| 12b | 1 | 8 | 1 | 16 | 1 | 16 |
| 12c | 0.5 | 4 | 0.5 | 8 | 1 | 8 |
| 12d | 0.5 | 8 | 1 | 16 | 2 | 16 |
| 12e | 0.5 | 8 | 1 | 16 | 1 | 8 |
| 13a | 0.5 | 8 | 1 | 16 | 2 | 8 |
| 13b | 1 | 8 | 1 | 16 | 2 | 16 |
| 13c | 0.5 | 4 | 1 | 16 | 1 | 8 |
| 13d | 1 | 8 | 1 | 16 | 2 | 16 |
| 13e | 1 | 8 | 1 | 16 | 2 | 16 |

### Example 4

A dose-response study was performed on seven compounds in combination with colistin against the three strains that contain the mcr-1 plasmid (Table 4). The concentration of the adjuvant was sequentially lowered to determine the point at which the colistin MIC exceeded the breakpoint for clinical colistin susceptibility (2 μg/mL). All adjuvants maintained similar activity to the original 50 μM dose when tested at 30 μM, and adjuvants 8b, 8c, 8e, 9d, and 9e successfully lowered the colistin MIC to the susceptibility breakpoint at 15 μM against all three strains. Compound 9d was the second most potent adjuvant and lowered the colistin MIC to 1 μg/mL against all mcr-1 strains at 15 μM, while adjuvant 9e displayed significant potentiation activity down to 5 μM, where it reduced the MIC of colistin from 8, 16, and 16 to 1, 2, and 1 μg/mL against mcr-1 plasmid containing E. coli, K. pneumoniae, and A. baumannii, respectively.

TABLE 4
Dose response of six lead compounds with colistin
(MIC μg/mL) against +mcr-l strains

| | Concentration | | | |
|---|---|---|---|---|
| | Tested | E. coli | K. pneumoniae | A. baumannii |
| Compound | (μM) | YD626^{+mcr-l} | 2210291^{+mcr-l} | 17978^{+mcr-l} |
| — | | 8 | 16 | 16 |
| 8b | 30 | 1 | 1 | 1 |
| | 20 | 2 | 1 | 1 |
| | 15 | 2 | 1 | 1 |
| | 10 | 4 | 2 | 2 |
| | 5 | — | 8 | 8 |
| 8c | 30 | 0.5 | 0.5 | 0.5 |
| | 20 | 1 | 1 | 1 |
| | 15 | 1 | 1 | 2 |
| | 10 | 4 | 4 | 4 |
| 8d | 30 | 1 | 1 | 1 |
| | 20 | 4 | 2 | 2 |
| | 15 | — | 4 | 4 |
| | 10 | — | 4 | |
| 8e | 30 | 1 | 1 | 1 |
| | 20 | 2 | 1 | 1 |
| | 15 | 2 | 2 | 2 |
| | 10 | 4 | 2 | 2 |
| | 5 | — | 4 | 8 |
| 9d | 30 | 1 | 1 | 1 |
| | 20 | 1 | 1 | 1 |
| | 15 | 1 | 1 | 1 |
| | 10 | 4 | 2 | 2 |
| | 5 | — | 4 | 4 |
| 9e | 30 | 1 | 0.5 | 0.5 |
| | 20 | 1 | 0.5 | 0.5 |
| | 15 | 1 | 0.5 | 0.5 |
| | 10 | 1 | 0.5 | 0.5 |
| | 5 | 1 | 2 | 1 |
| | 2.5 | 4 | 2 | 2 |
| | 1 | — | 16 | 16 |
| 11c | 30 | 0.25 | 1 | 1 |
| | 20 | 1 | 2 | 2 |
| | 15 | 1 | 4 | 4 |
| | 10 | 4 | — | — |

### Example 5

Compounds 8b, 9d, and 9e were selected to test against chromosomally colistin resistant K. pneumoniae and A. baumannii strains, which are typically significantly more resistant to colistin than isolates with resistance encoded by the mcr-1 gene. All three compounds were highly active at 20 μM, eliciting between 64 and ≥2048-fold reductions in the MIC of colistin (Table 5). Compound 9e was again the most active compound and resensitized all four chromosomally colistin resistant strains to or below the CLSI breakpoint (≤2 mg/mL) when tested at a concentration of 20 μM, and resensitized three out of the four strains to breakpoint colistin levels at a concentration of 7.5 μM. Against K. pneumoniae B9 and A. baumannii 4106, compound 9e returned over a 2000-fold reduction of the colistin MIC at 10 μM. Overall, these compounds, especially adjuvant 9e, displayed potent in vitro colistin potentiation activity against chromosomally resistant bacterial strains.

TABLE 5
Dose response of lead compounds 8b, 8d, and 9e with colistin
(MIC μg/mL) against chromosomally colistin resistant strains.

| | | K. | K. | A. | A. |
|---|---|---|---|---|---|
| | Concentration | pneumoniae | pneumoniae | baumannii | baumannii |
| Compound | (μM) | B9 | C3 | 3941 | 4106 |
| * | | 512 | 512 | 512 | 1024 |
| 8b | 20 | [symbol] 0.25 | 2 | 8 | 4 |
| | 15 | 0.5 | 4 | 8 | 4 |
| | 10 | 1 | 8 | 16 | 8 |
| | 7.5 | 4 | — | — | — |
| | 8 | 32 | — | — | — |
| 9d | 20 | [symbol] 0.25 | 1 | 2 | 2 |
| | 15 | 0.5 | 2 | 4 | 2 |
| | 10 | 1 | 4 | 8 | 4 |
| | 7.5 | 2 | — | — | — |
| | 5 | 8 | — | — | — |
| 9e | 20 | [symbol] 0.25 | 0.5 | 2 | 0.5 |
| | 15 | [symbol] 0.25 | 0.5 | 4 | 0.5 |
| | 10 | [symbol] 0.25 | 1 | 4 | 1 |
| | 7.5 | [symbol] 0.25 | 2 | 16 | 1 |
| | 8 | [symbol] 0.5 | 2 | — | 4 |

[symbol]

indicates data missing or illegible when filed

### Example 6

Additional assays were performed with compound 9e to confirm activity and examine toxicity. To rule out activity due to nonspecific compound aggregation effects, the resensitization of A. baumannii 17978^{+mcr-1} to colistin was performed in the presence of Tween 80. Compound 9e displayed equipotent colistin sensitization activity in the absence and presence of Tween 80 when tested at 30 μM. Next, adjuvant 9e and compound 9a were tested for toxicity against red blood cells at a concentration of 200 μM, and both compounds effected <2.5% cell lysis. Lastly, the potential for eukaryotic cell toxicity was explored using a mouse mammary gland tumor cell line, 4T1 (ATCC). Five analogs (8b, 8c, 8d, 9d, and 9e), in addition to 14 were each tested in mono-therapy

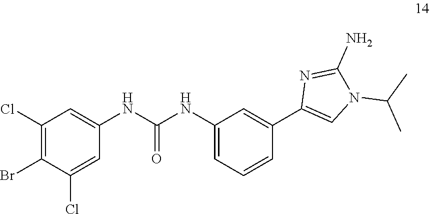 [SMILES: CC(C)n1cc(-c2cccc(NC(=O)Nc3cc(Cl)c(Br)c(Cl)c3)c2)nc1N]

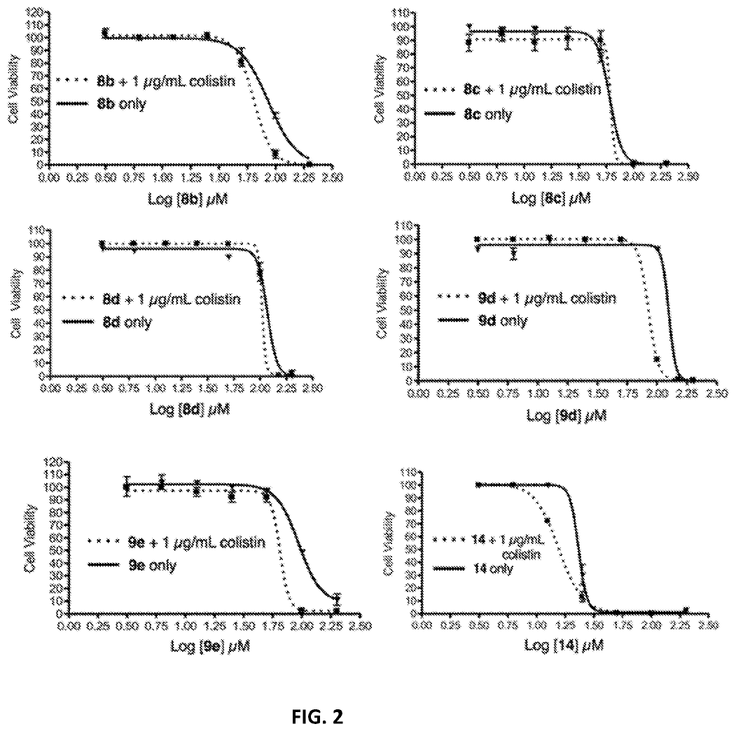

and in combination with colistin (1 μg/mL). After 18 hours of incubation with 4T1 cells, the culture medium was aspirated and cells were treated with a 0.5 mg/mL solution of 3-(4,5-dimethylthiazol-2-yl)-2,5-diphenyltetrazolium bromide (MTT) in culture media to afford a colorimetric assay for cell viability (FIG. 2). The concentration at which each compound resulted in 50% cell viability was recorded as that compound's CT₅₀ value (Table 6). From these data, the respective CT₅₀ value of each compound was not significantly altered by the presence of colistin in culture media. Second, each compound was considerably less toxic than 14.

TABLE 6
CT50 (μM) of analogs in 4T1 cells dosed
with and without 1 μg/mL colistin

| | Compound alone | Compound + colistin |
|---|---|---|
| 8b | 86.2 | 64.2 |
| 8c | 60.5 | 61.8 |
| 8d | 117.3 | 104.8 |
| 9d | 125.5 | 84.6 |
| 9e | 94.1 | 64.9 |
| 14 | 23.2 | 15.6 |

### Example 7

An antibiotic-adjuvant combination was next tested in an in vivo model: a Galleria mellonella model of A. baumannii infection. In previous studies at the Walter Reed Army Institute of Research (WRAIR), results from this model have been shown to be predictive of outcomes in murine models of infection (Jacobs et al., mBio 2014, 5 (3), e01076-14; Huggins et al., ACS Med. Chem. Lett. 2017, 8(1), 27-31).

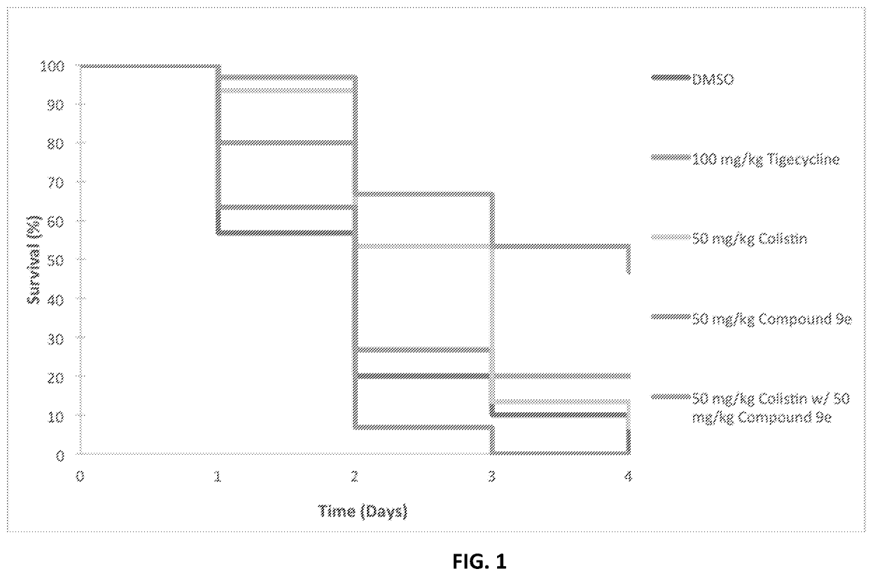

A dose escalation study was first performed using A. baumannii 4106 in a G. mellonella to determine the minimum lethal dose of the bacteria in the worms. This strain was notably virulent, and 6×10⁵ colony forming units (CFUs) brought about 100% death of moth larva by day 4, which is similar to the highly virulent A. baumannii 5075 strain. This strain was essentially resistant to all antibiotic classes, with the antibiotic tigecycline demonstrating the most potent in vitro MIC of 8 μg/mL. With this antibiotic as positive control, a dose of 100 mg/kg provided minimal survival after 4 days (20% survival at day 4, FIG. 1).

Treatment of infected worms with either 50 mg/kg colistin or compound 9e at 50 mg/kg provided only 7% and 0% survival respectively after 4 days. However, when 50 mg/kg colistin was paired with 50 mg/kg compound 9e, the combination increased worm survival to 47% after 4 days, considerably exceeding that of the tigecycline control (FIG. 1).

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A method of treating a gram-negative bacterial infection comprising administering to a subject in need thereof, a polymyxin antibiotic, or a pharmaceutically acceptable salt thereof, and a compound of formula (I) in amounts that together are effective to inhibit the gram-negative bacterial infection,

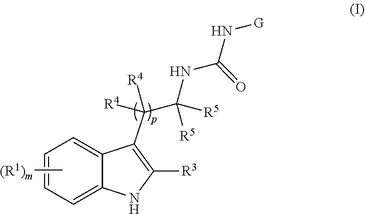

wherein
R¹ is halogen, cyano, CH₃, OCH₃, OCF₃, or OCHF₂;
G is a 6- to 12-membered aryl optionally substituted with 1-5 R² substituents;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, or —OC₃₋₆cycloalkyl;
R³ is hydrogen, C₁₋₄alkyl, C₁₋₄haloalkyl, or C₃₋₆cycloalkyl;
R⁴, at each occurrence, is independently hydrogen, C₁₋₄alkyl, C₁₋₄haloalkyl, or C₃₋₆cycloalkyl;
R⁵, at each occurrence, is independently hydrogen, C₁₋₄alkyl, C₁₋₄haloalkyl, or C₃₋₆cycloalkyl;
p is 0 or 1; and
m is 0, 1, or 2.

Clause 2. A method of potentiating the activity of a polymyxin antibiotic against a gram-negative bacterial infection comprising administering to a subject in need thereof a compound of formula (I) in an amount effective to increase the therapeutic effect of a dose of the polymyxin antibiotic, or a pharmaceutically acceptable salt thereof, compared to the therapeutic effect of the dose of the polymyxin antibiotic, or a pharmaceutically acceptable salt thereof, in the absence of the compound of formula (I),

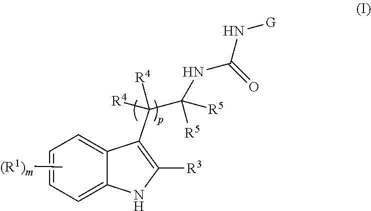

wherein
R¹ is halogen, cyano, CH₃, OCH₃, OCF₃, or OCHF₂;
G is a 6- to 12-membered aryl optionally substituted with 1-5 R² substituents;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, or —OC₃₋₆cycloalkyl;
R³ is hydrogen, C₁₋₄alkyl, C₁₋₄haloalkyl, or C₃₋₆cycloalkyl;
R⁴, at each occurrence, is independently hydrogen, C₁₋₄alkyl, C₁₋₄haloalkyl, or C₃₋₆cycloalkyl;
R⁵, at each occurrence, is independently hydrogen, C₁₋₄alkyl, C₁₋₄haloalkyl, or C₃₋₆cycloalkyl;
p is 0 or 1; and
m is 0, 1, or 2.

Clause 3. The method of clause 1 or 2, wherein the gram-negative bacterial infection is a multidrug-resistant gram-negative bacterial infection.

Clause 4. The method of any of clauses 1-3, wherein the gram-negative bacterial infection is an infection of a bacteria species selected from one or more of E. coli, K. pneumonia, and A. baumannii.

Clause 5. The method of any of clauses 1-4, wherein the gram-negative bacterial infection is an infection of a bacterial strain possessing an mcr gene.

Clause 6. The method of any of clauses 1-5, wherein the polymyxin antibiotic is colistin, colistimethate, or a pharmaceutically acceptable salt thereof.

Clause 7. The method of any of clauses 1-6, wherein the compound of formula (I) has formula (II)

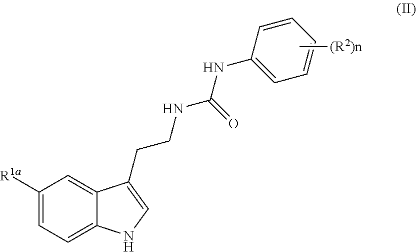

wherein
R^{1a} is hydrogen, halogen, cyano, CH₃, OCH₃, OCF₃, or OCHF₂; and
n is 0, 1, 2, 3, 4, or 5.

Clause 8. The method of clause 7, wherein the compound of formula (II) has one of the following formulas:

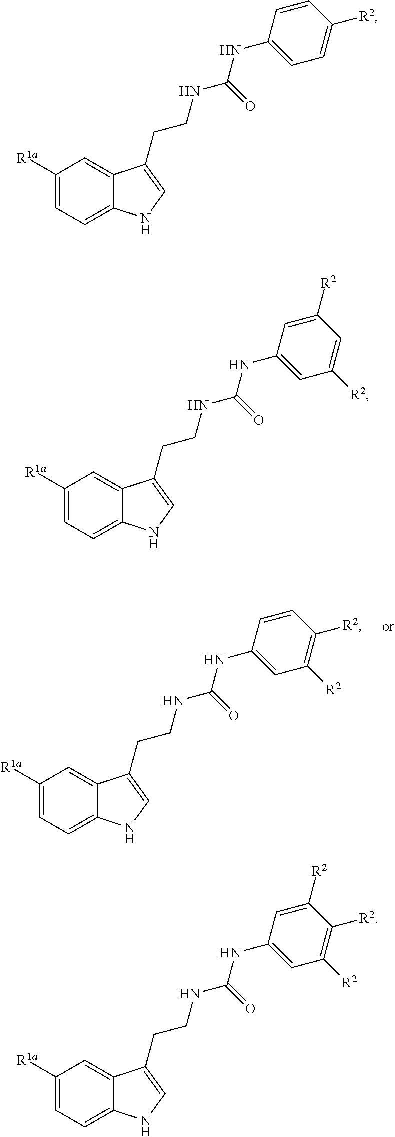

Clause 9 The method of clause 8, wherein the compound of formula (II) has one of the following formulas:

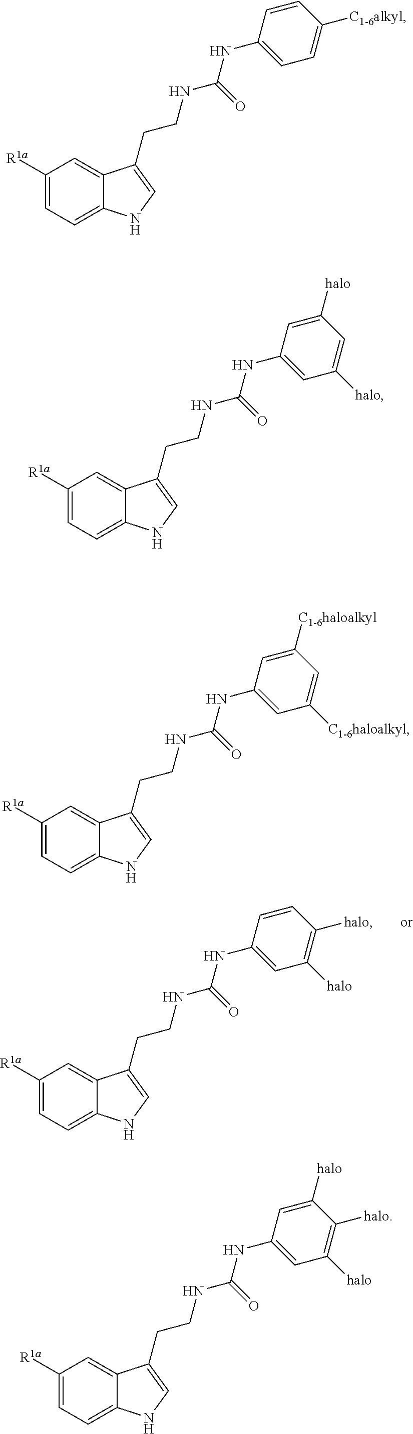

Clause 10. The method of clause 9, wherein the compound of formula (II) has one of the following formulas:

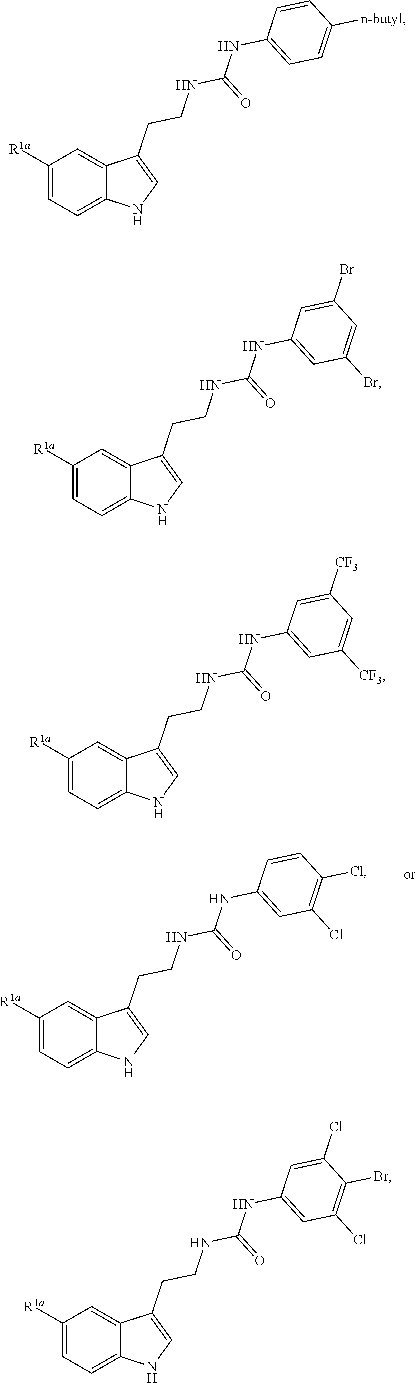

Clause 11. The method of any of clauses 1-6, wherein the compound of formula (I) has formula (III)

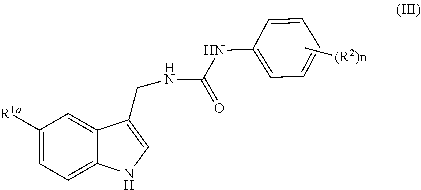

wherein
R^{1a} is hydrogen, halogen, cyano, CH₃, OCH₃, OCF₃, or OCHF₂; and
n is 0, 1, 2, 3, 4, or 5.

Clause 12. The method of clause 11, wherein the compound of formula (III) has one of the following formulas:

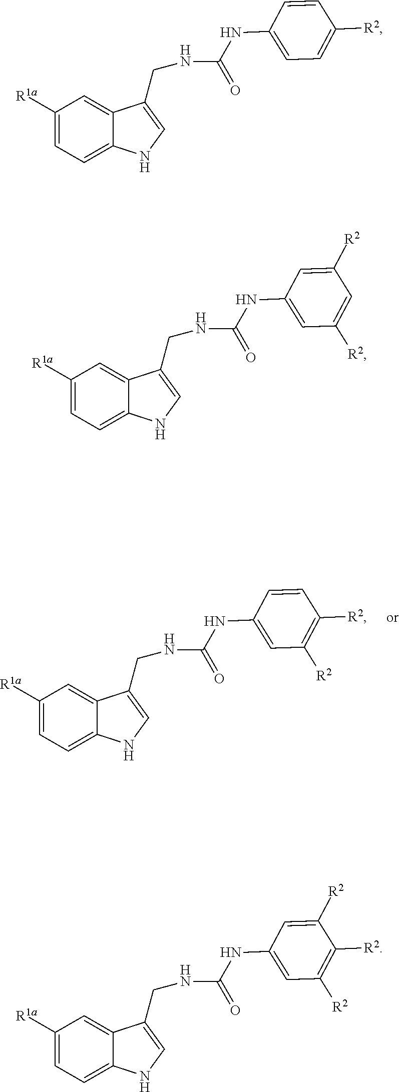

Clause 13. The method of clause 12, wherein the compound of formula (III) has one of the following formulas:

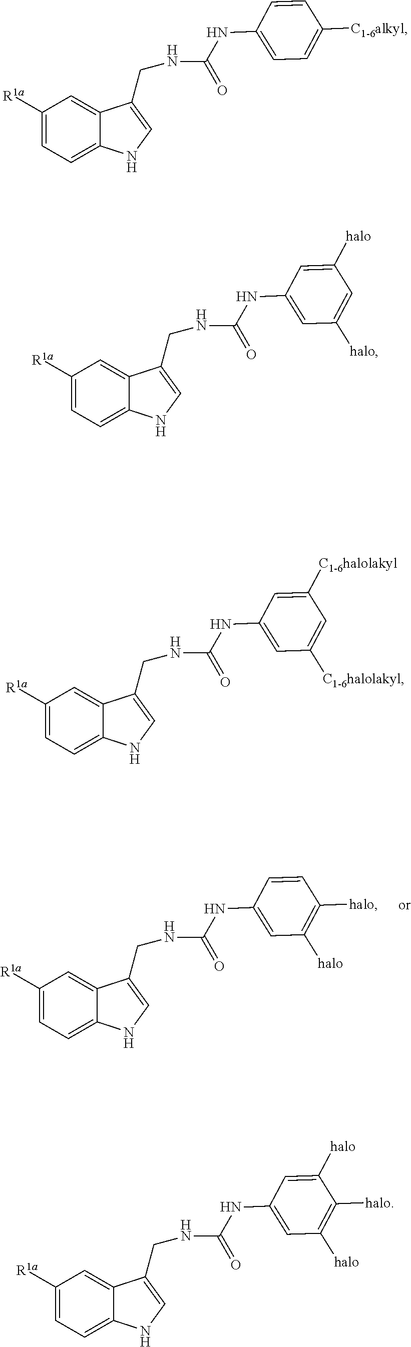

Clause 14. The method of clause 13, wherein the compound of formula (III) has one of the following formulas:

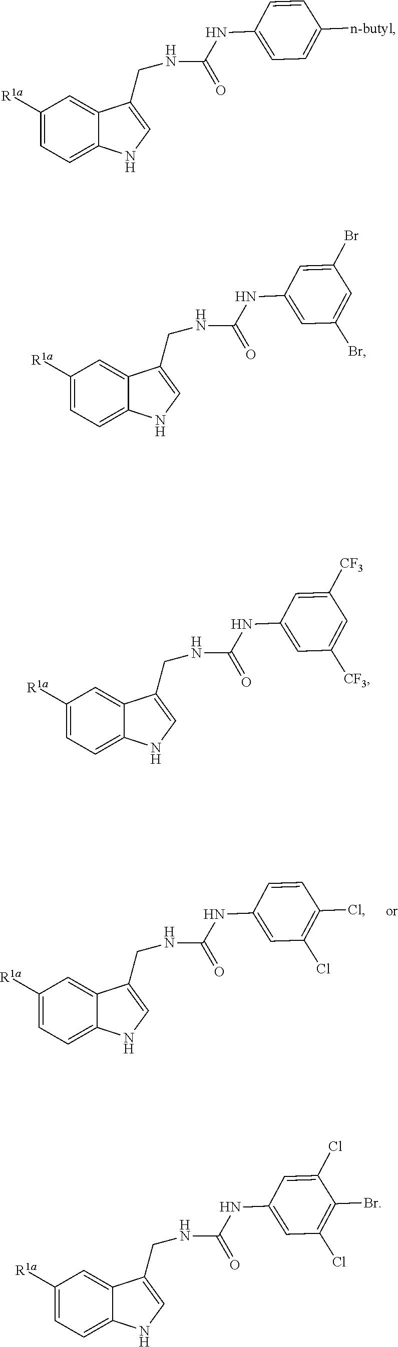

Clause 15. The method of any of clauses 7-14, wherein R^{1a} is hydrogen.

Clause 16. The method of any of clauses 7-14, wherein R^{1a} is halogen.

Clause 17. The method of any of clauses 1-6, wherein the compound of formula (I) is selected from the group consisting of:
1-(2-(1H-indol-3-yl)ethyl)-3-(4-butylphenyl)urea;
1-(2-(1H-indol-3-yl)ethyl)-3-(3,5-dibromophenyl)urea;
1-(2-(1H-indol-3-yl)ethyl)-3-(3,5-bis(trifluoromethyl)phenyl)urea;
1-(2-(1H-indol-3-yl)ethyl)-3-(3,4-dichlorophenyl)urea;
1-(2-(1H-indol-3-yl)ethyl)-3-(4-bromo-3,5-dichlorophenyl)urea;
1-(2-(5-bromo-1H-indol-3-yl)ethyl)-3-(4-butylphenyl)urea;
1-(2-(5-bromo-1H-indol-3-yl)ethyl)-3-(3,5-dibromophenyl)urea;
1-(3,5-bis(trifluoromethyl)phenyl)-3-(2-(5-bromo-1H-indol-3-yl)ethyl)urea;
1-(2-(5-bromo-1H-indol-3-yl)ethyl)-3-(3,4-dichlorophenyl)urea;
1-(2-(5-bromo-1H-indol-3-yl)ethyl)-3-(4-bromo-3,5-dichlorophenyl)urea;
1((1H-indol-3-yl)methyl)-3-(4-butylphenyl)urea;
1((1H-indol-3-yl)methyl)-3-(3,5-dibromophenyl)urea;
1((1H-indol-3-yl)methyl)-3-(3,5-bis(trifluoromethyl)phenyl)urea;
1((1H-indol-3-yl)methyl)-3-(3,4-dichlorophenyl)urea; and
1((1H-indol-3-yl)methyl)-3-(4-bromo-3,5-dichlorophenyl)urea.

Clause 18. A compound of formula (IV)

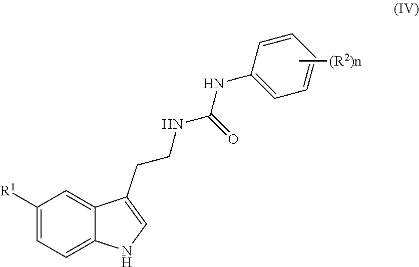

wherein
R¹ is halogen, cyano, CH₃, OCH₃, OCF₃, or OCHF₂;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, —OC₃₋₆cycloalkyl; and
n is 0, 1, 2, 3, 4, or 5;
provided that the compound of formula (IV) is not:
N-[2-(5-fluoro-1H-indol-3-yl)ethyl]-N′-(4-methoxyphenyl)-urea;
N-(2-fluorophenyl)-N′-[2-(5-methyl-1Hindol-3-yl)ethyl]-urea;
N-(3-fluorophenyl)-N′-[2-(5-methyl-1Hindol-3-yl)ethyl]-urea;
N-(4-ethoxyphenyl)-N′-[2-(6-methoxy-1Hindol-3-yl)ethyl]-urea;
N-[2-(5-chloro-1H-indol-3-yl)ethyl]-N′-(2,4-dimethoxyphenyl)-urea;
N-(3,5-dimethoxyphenyl)-N′-[2-(5-fluoro-1H-indol-3-yl)ethyl]-urea;
N-[2-(5-fluoro-1H-indol-3-yl)ethyl]-N′-(3-methylphenyl)-urea;
N-(5-chloro-2-methoxyphenyl)-N′-[2-(5-fluoro-1H-indol-3-yl)ethyl]-urea;
N-(3-bromophenyl)-N′-[2-(5-fluoro-1Hindol-3-yl)ethyl]-urea;
N-(3-chlorophenyl)-N′-[2-(5-fluoro-1Hindol-3-yl)ethyl]-urea;
N-(4-chlorophenyl)-N′-[2-(5-fluoro-1Hindol-3-yl)ethyl]-urea;
N-(3,4-dichlorophenyl)-N′-[2-(5-fluoro-1Hindol-3-yl)ethyl]-urea;
N-[2-(5-fluoro-1H-indol-3-yl)ethyl]-N′-(4-methylphenyl)-urea;
N-[2-(5-fluoro-1H-indol-3-yl)ethyl]-N′-phenyl-urea;
N-[2-(5-chloro-1H-indol-3-yl)ethyl]-N′-(4-methylphenyl)-urea;
N-[2-(5-methyl-1H-indol-3-yl)ethyl]-N′-(4-methylphenyl)-urea;
N-[2-(5-chloro-1H-indol-3-yl)ethyl]-N′-(4-fluorophenyl)-urea;
N-[2-(5-chloro-1H-indol-3-yl)ethyl]-N′-(4-nitrophenyl)-urea;
N-[2-(5-chloro-1H-indol-3-yl)ethyl]-N′-(3-chlorophenyl)-urea;
N-[2-(5-chloro-1H-indol-3-yl)ethyl]-N′-(3,4-dichlorophenyl)-urea;
N-[2-(5-chloro-1H-indol-3-yl)ethyl]-N′-(4-chlorophenyl)-urea;
N-[2-(5-chloro-1H-indol-3-yl)ethyl]-N′-phenyl-urea;
N-(4-fluorophenyl)-N′-[2-(5-methyl-1Hindol-3-yl)ethyl]-urea;
N-[2-(5-methyl-1H-indol-3-yl)ethyl]-N′-(4-nitrophenyl)-urea;
N-(3-chlorophenyl)-N′-[2-(5-methyl-1Hindol-3-yl)ethyl]-urea;
N-(3,4-dichlorophenyl)-N′-[2-(5-methyl-1Hindol-3-yl)ethyl]-urea;
N-(4-chlorophenyl)-N′-[2-(5-methyl-1Hindol-3-yl)ethyl]-urea; or
N-[2-(5-methyl-1H-indol-3-yl)ethyl]-N′-phenyl-urea.

Clause 19. The compound of clause 18 of formula

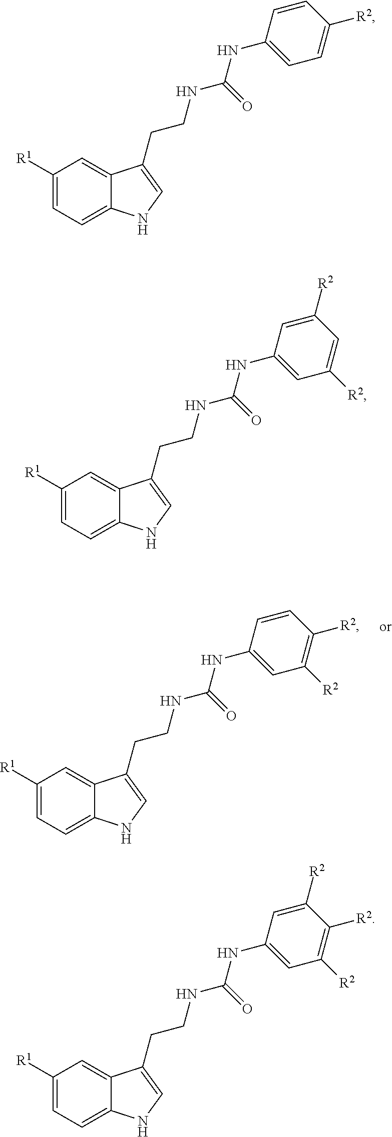

Clause 20. The compound of clause 19 of formula

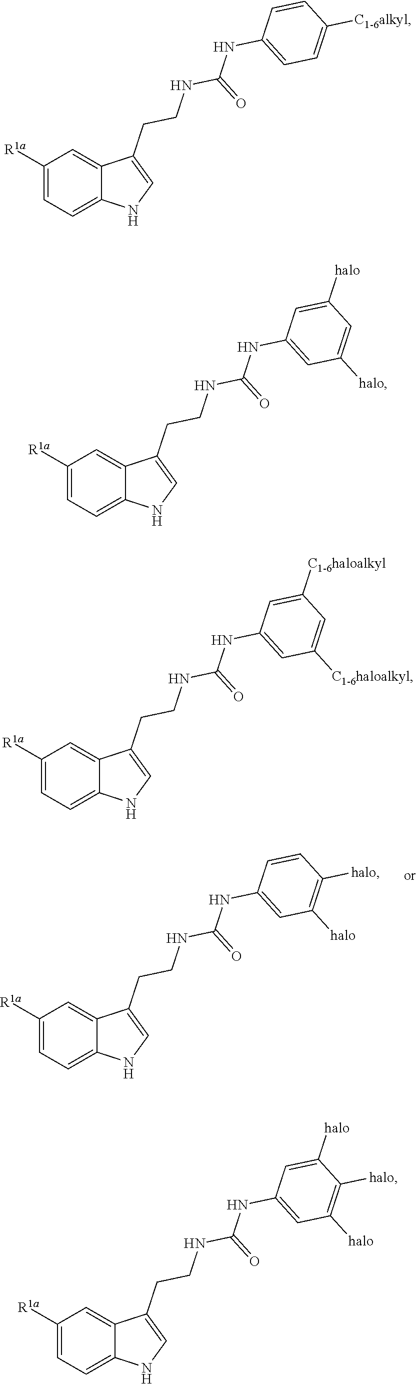

Clause 21. The compound of clause 20 of formula

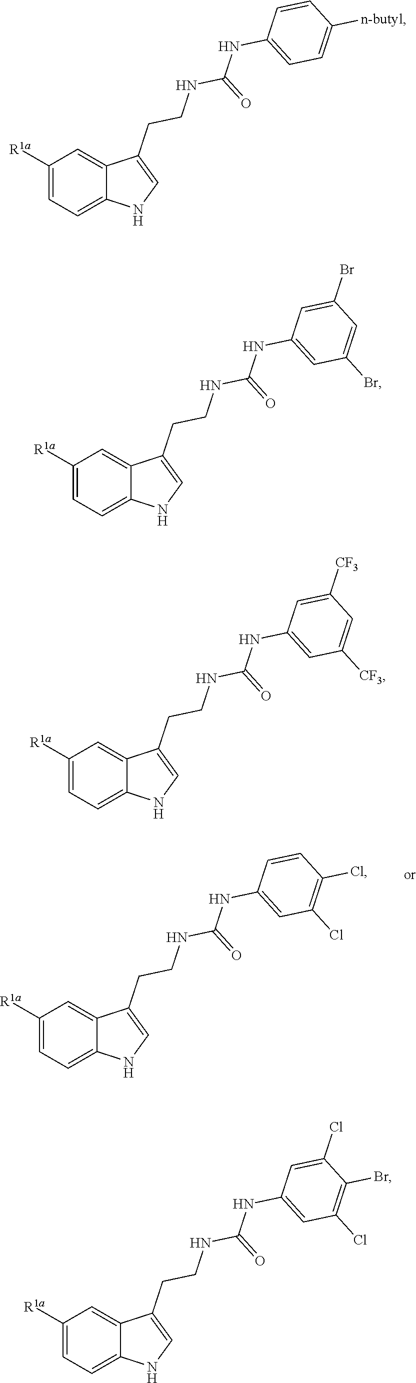

Clause 22. A compound of formula (III)

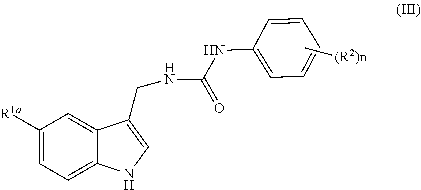

wherein
R^{1a} is hydrogen, halogen, cyano, CH₃, OCH₃, OCF₃, or OCHF₂;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, or —OC₃₋₆cycloalkyl; and
n is 0, 1, 2, 3, 4, or 5.

Clause 23. The compound of clause 22 of formula

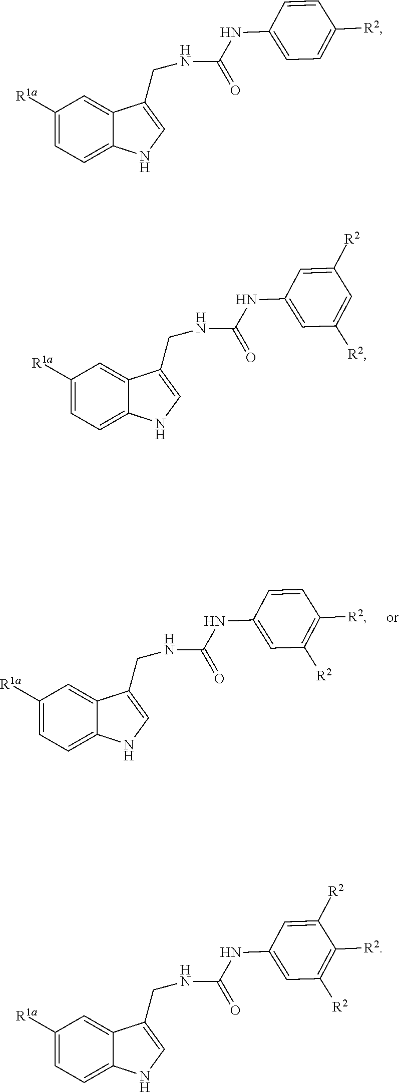

Clause 24. The compound of clause 23 of formula

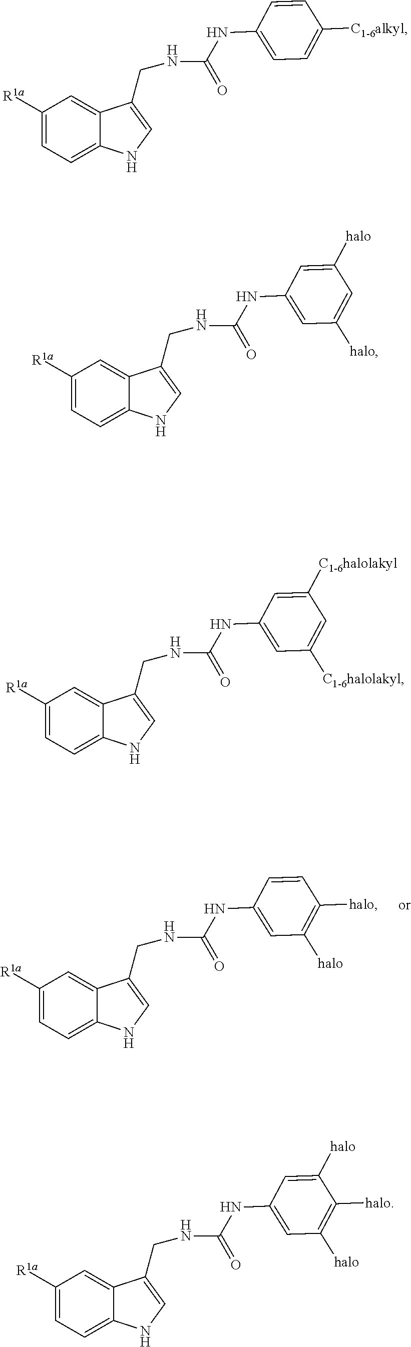

Clause 25. The compound of clause 24 of formula

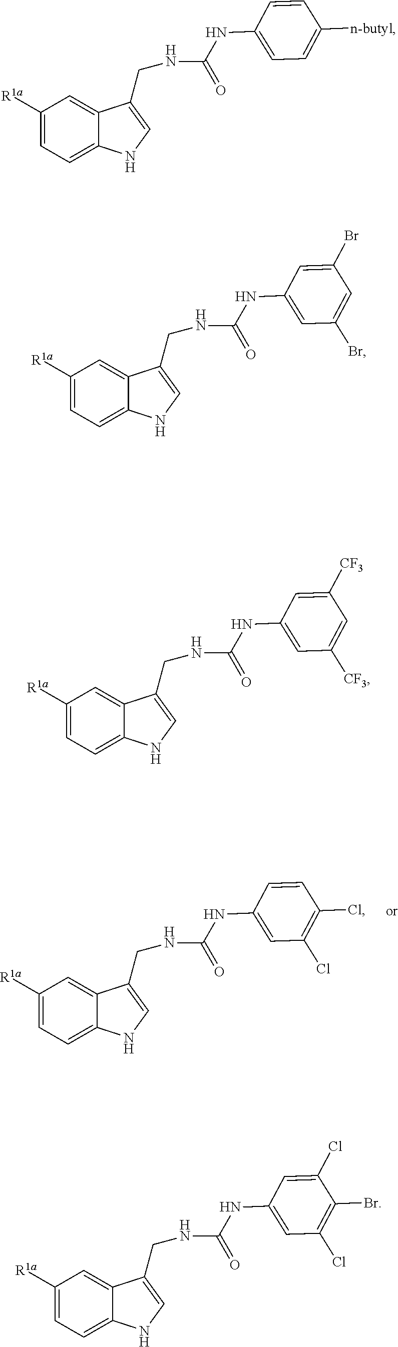

Clause 26. The compound of any of clauses 18-21, wherein R¹ is halogen.

Clause 27. The compound of any of clauses 22-25, wherein R^{1a} is hydrogen.

Clause 28. The compound of any of clauses 22-25, wherein R^{1a} is halogen.

Clause 29. A compound selected from the group consisting of:
1-(2-(1H-indol-3-yl)ethyl)-3-(3,5-dibromophenyl)urea;
1-(2-(1H-indol-3-yl)ethyl)-3-(4-bromo-3,5-dichlorophenyl)urea;
1-(2-(5-bromo-1H-indol-3-yl)ethyl)-3-(4-butylphenyl)urea;
1-(2-(5-bromo-1H-indol-3-yl)ethyl)-3-(3,5-dibromophenyl)urea;
1-(3,5-bis(trifluoromethyl)phenyl)-3-(2-(5-bromo-1H-indol-3-yl)ethyl)urea;
1-(2-(5-bromo-1H-indol-3-yl)ethyl)-3-(3,4-dichlorophenyl)urea;
1-(2-(5-bromo-1H-indol-3-yl)ethyl)-3-(4-bromo-3,5-dichlorophenyl)urea;
1((1H-indol-3-yl)methyl)-3-(4-butylphenyl)urea;
1((1H-indol-3-yl)methyl)-3-(3,5-dibromophenyl)urea;
1((1H-indol-3-yl)methyl)-3-(3,5-bis(trifluoromethyl)phenyl)urea;
1((1H-indol-3-yl)methyl)-3-(3,4-dichlorophenyl)urea; and
1((1H-indol-3-yl)methyl)-3-(4-bromo-3,5-dichlorophenyl)urea.

Clause 30. A pharmaceutical composition comprising the compound of any of clauses 18-29, and a pharmaceutically acceptable carrier.

1. A method of treating a gram-negative bacterial infection comprising administering to a subject in need thereof, a polymyxin antibiotic, or a pharmaceutically acceptable salt thereof, and a compound of formula (I) in amounts that together are effective to inhibit the gram-negative bacterial infection, 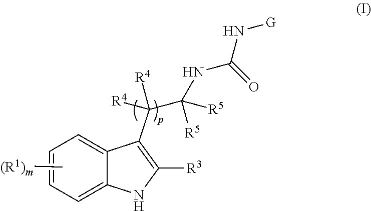
wherein
R¹ is halogen, cyano, CH₃, OCH₃, OCF₃, or OCHF₂;
G is a 6- to 12-membered aryl optionally substituted with 1-5 R² substituents;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, or —OC₃₋₆cycloalkyl;
R³ is hydrogen, C₁₋₄alkyl, C₁₋₄haloalkyl, or C₃₋₆cycloalkyl;
R⁴, at each occurrence, is independently hydrogen, C₁₋₄alkyl, C₁₋₄haloalkyl, or C₃₋₆cycloalkyl;
R⁵, at each occurrence, is independently hydrogen, C₁₋₄alkyl, C₁₋₄haloalkyl, or C₃₋₆cycloalkyl;
p is 0 or 1; and
m is 0, 1, or 2.

2. A method of potentiating the activity of a polymyxin antibiotic against a gram-negative bacterial infection comprising administering to a subject in need thereof a compound of formula (I) in an amount effective to increase the therapeutic effect of a dose of the polymyxin antibiotic, or a pharmaceutically acceptable salt thereof, compared to the therapeutic effect of the dose of the polymyxin antibiotic, or a pharmaceutically acceptable salt thereof, in the absence of the compound of formula (I), 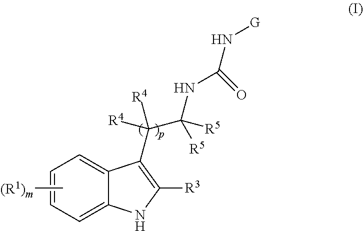
wherein
R¹ is halogen, cyano, CH₃, OCH₃, OCF₃, or OCHF₂;
G is a 6- to 12-membered aryl optionally substituted with 1-5 R² substituents;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, or —OC₃₋₆cycloalkyl;
R³ is hydrogen, C₁₋₄alkyl, C₁₋₄haloalkyl, or C₃₋₆cycloalkyl;
R⁴, at each occurrence, is independently hydrogen, C₁₋₄alkyl, C₁₋₄haloalkyl, or C₃₋₆cycloalkyl;
R⁵, at each occurrence, is independently hydrogen, C₁₋₄alkyl, C₁₋₄haloalkyl, or C₃₋₆cycloalkyl;
p is 0 or 1; and
m is 0, 1, or 2.

3. The method of claim 1, wherein the gram-negative bacterial infection is a multidrug-resistant gram-negative bacterial infection.

4. The method of claim 3, wherein the gram-negative bacterial infection is an infection of a bacteria species selected from one or more of E. coli, K. pneumonia, and A. baumannii.

5. The method of claim 4, wherein the gram-negative bacterial infection is an infection of a bacterial strain possessing an mcr gene.

6. The method of claim 3, wherein the polymyxin antibiotic is colistin, colistimethate, or a pharmaceutically acceptable salt thereof.

7. The method of claim 6, wherein the compound of formula (I) has formula (II) 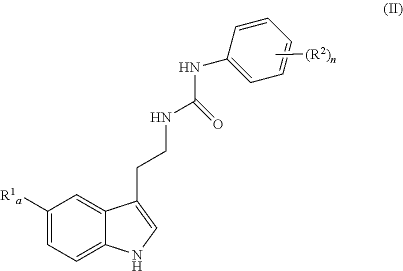
wherein
R^{1a} is hydrogen, halogen, cyano, CH₃, OCH₃, OCF₃, or OCHF₂; and
n is 0, 1, 2, 3, 4, or 5.

8. The method of claim 7, wherein the compound of formula (II) has one of the following formulas:

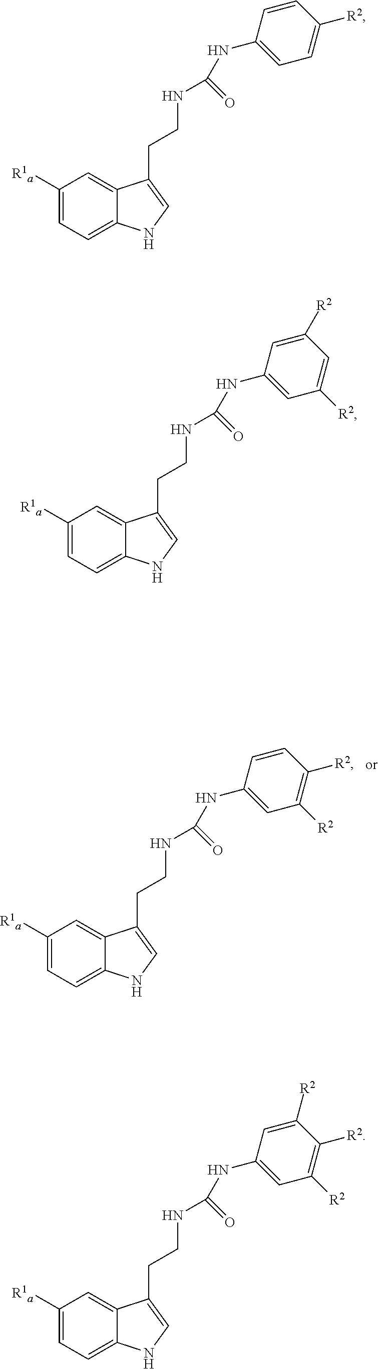

9. The method of claim 8, wherein the compound of formula (II) has one of the following formulas:

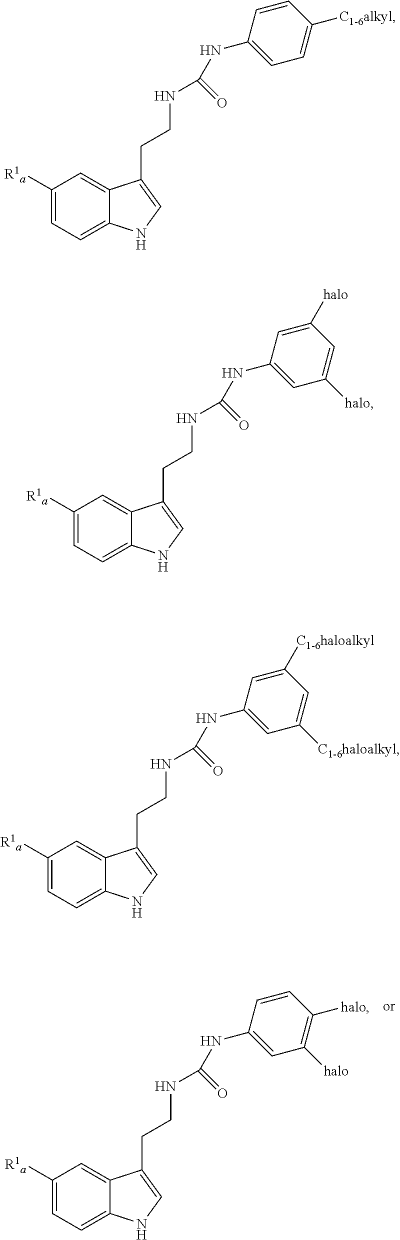

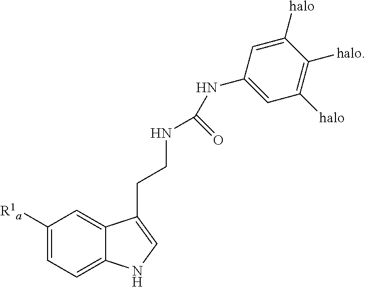

10. The method of claim 9, wherein the compound of formula (II) has one of the following formulas:

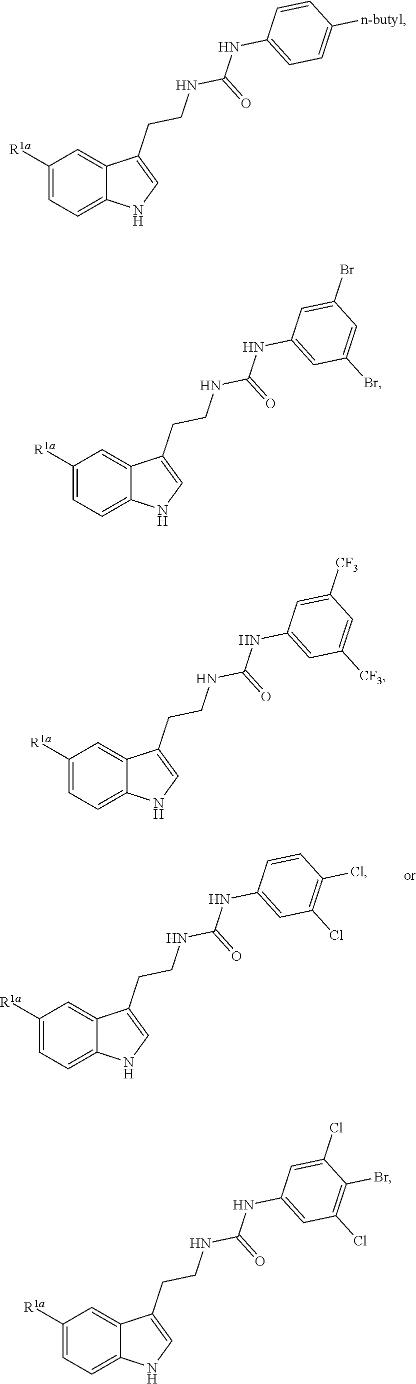

11. The method of claim 6, wherein the compound of formula (I) has formula (III) 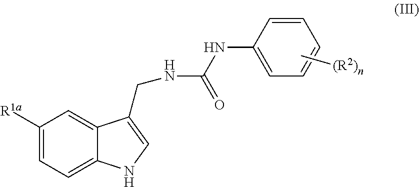
wherein
R^{1a} is hydrogen, halogen, cyano, CH₃, OCH₃, OCF₃, or OCHF₂; and
n is 0, 1, 2, 3, 4, or 5.

12. The method of claim 11, wherein the compound of formula (III) has one of the following formulas:

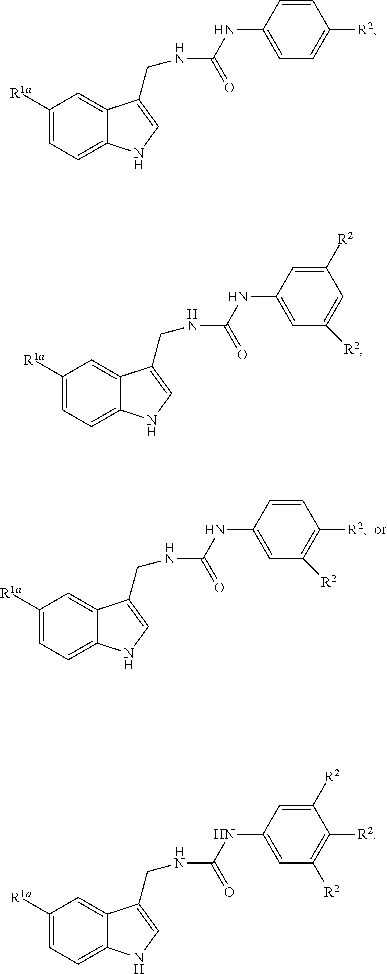

13. The method of claim 12, wherein the compound of formula (III) has one of the following formulas:

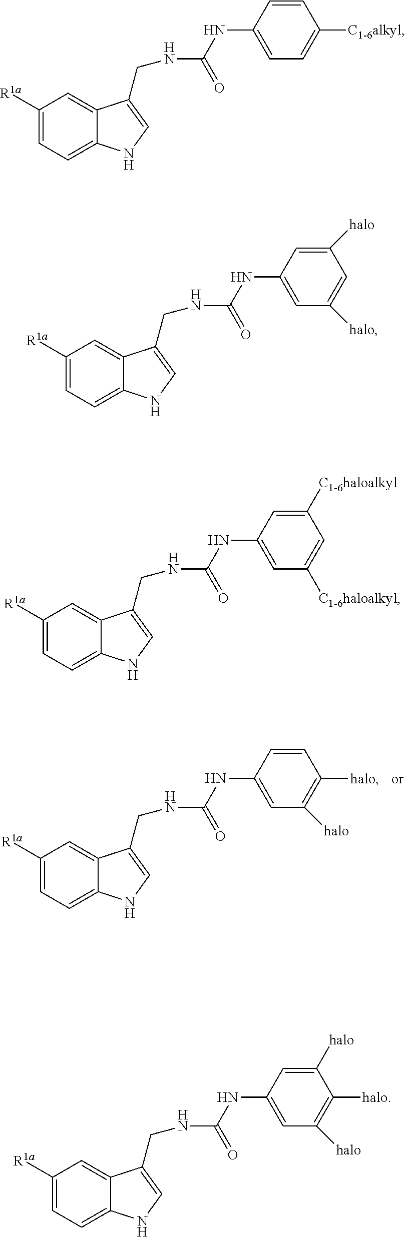

14. The method of claim 13, wherein the compound of formula (III) has one of the following formulas:

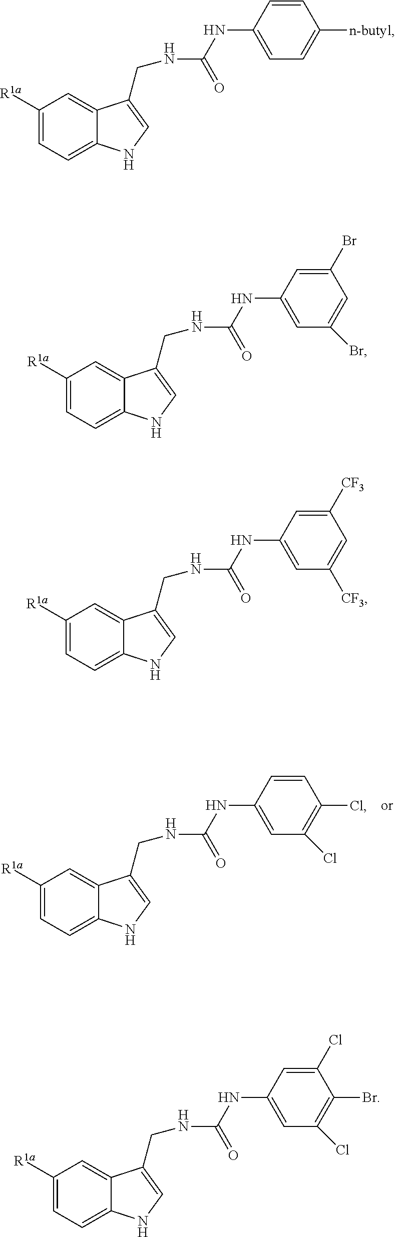

15. The method of claim 13, wherein R^{1a} is hydrogen.

16. The method of claim 13, wherein R^{1a} is halogen.

17. The method of claim 6, wherein the compound of formula (I) is selected from the group consisting of:
1-(2-(1H-indol-3-yl)ethyl)-3-(4-butylphenyl)urea;
1-(2-(1H-indol-3-yl)ethyl)-3-(3,5-dibromophenyl)urea;
1-(2-(1H-indol-3-yl)ethyl)-3-(3,5-bis(trifluoromethyl)phenyl)urea;
1-(2-(1H-indol-3-yl)ethyl)-3-(3,4-dichlorophenyl)urea;
1-(2-(1H-indol-3-yl)ethyl)-3-(4-bromo-3,5-dichlorophenyl)urea;
1-(2-(5-bromo-1H-indol-3-yl)ethyl)-3-(4-butylphenyl)urea;
1-(2-(5-bromo-1H-indol-3-yl)ethyl)-3-(3,5-dibromophenyl)urea;
1-(3,5-bis(trifluoromethyl)phenyl)-3-(2-(5-bromo-1H-indol-3-yl)ethyl)urea;
1-(2-(5-bromo-1H-indol-3-yl)ethyl)-3-(3,4-dichlorophenyl)urea;
1-(2-(5-bromo-1H-indol-3-yl)ethyl)-3-(4-bromo-3,5-dichlorophenyl)urea;
1((1H-indol-3-yl)methyl)-3-(4-butylphenyl)urea;
1((1H-indol-3-yl)methyl)-3-(3,5-dibromophenyl)urea;
1((1H-indol-3-yl)methyl)-3-(3,5-bis(trifluoromethyl)phenyl)urea;
1((1H-indol-3-yl)methyl)-3-(3,4-dichlorophenyl)urea; and
1((1H-indol-3-yl)methyl)-3-(4-bromo-3,5-dichlorophenyl)urea.

18. A compound of formula (IV) 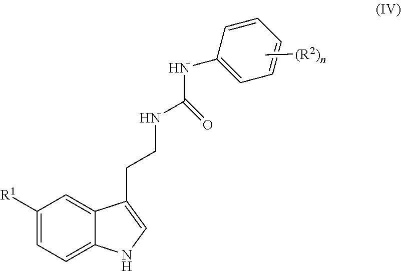
wherein
R¹ is halogen, cyano, CH₃, OCH₃, OCF₃, or OCHF₂;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, —OC₃₋₆cycloalkyl; and
n is 0, 1, 2, 3, 4, or 5;
provided that the compound of formula (IV) is not:
N-[2-(5-fluoro-1H-indol-3-yl)ethyl]-N′-(4-methoxyphenyl)-urea;
N-(2-fluorophenyl)-N′-[2-(5-methyl-1Hindol-3-yl)ethyl]-urea;
N-(3-fluorophenyl)-N′-[2-(5-methyl-1Hindol-3-yl)ethyl]-urea;
N-(4-ethoxyphenyl)-N′-[2-(6-methoxy-1Hindol-3-yl)ethyl]-urea;
N-[2-(5-chloro-1H-indol-3-yl)ethyl]-N′-(2,4-dimethoxyphenyl)-urea;
N-(3,5-dimethoxyphenyl)-N′-[2-(5-fluoro-1H-indol-3-yl)ethyl]-urea;
N-[2-(5-fluoro-1H-indol-3-yl)ethyl]-N′-(3-methylphenyl)-urea;
N-(5-chloro-2-methoxyphenyl)-N-[2-(5-fluoro-1H-indol-3-yl)ethyl]-urea;
N-(3-bromophenyl)-N′-[2-(5-fluoro-1Hindol-3-yl)ethyl]-urea;
N-(3-chlorophenyl)-N′-[2-(5-fluoro-1Hindol-3-yl)ethyl]-urea;
N-(4-chlorophenyl)-N′-[2-(5-fluoro-1Hindol-3-yl)ethyl]-urea;
N-(3,4-dichlorophenyl)-N′-[2-(5-fluoro-1Hindol-3-yl)ethyl]-urea;
N-[2-(5-fluoro-1H-indol-3-yl)ethyl]-N′-(4-methylphenyl)-urea;
N-[2-(5-fluoro-1H-indol-3-yl)ethyl]-N′-phenyl-urea;
N-[2-(5-chloro-1H-indol-3-yl)ethyl]-N′-(4-methylphenyl)-urea;
N-[2-(5-methyl-1H-indol-3-yl)ethyl]-N′-(4-methylphenyl)-urea;
N-[2-(5-chloro-1H-indol-3-yl)ethyl]-N′-(4-fluorophenyl)-urea;
N-[2-(5-chloro-1H-indol-3-yl)ethyl]-N′-(4-nitrophenyl)-urea;
N-[2-(5-chloro-1H-indol-3-yl)ethyl]-N′-(3-chlorophenyl)-urea;
N-[2-(5-chloro-1H-indol-3-yl)ethyl]-N′-(3,4-dichlorophenyl)-urea;
N-[2-(5-chloro-1H-indol-3-yl)ethyl]-N′-(4-chlorophenyl)-urea;
N-[2-(5-chloro-1H-indol-3-yl)ethyl]-N′-phenyl-urea;
N-(4-fluorophenyl)-N′-[2-(5-methyl-1Hindol-3-yl)ethyl]-urea;
N-[2-(5-methyl-1H-indol-3-yl)ethyl]-N′-(4-nitrophenyl)-urea;
N-(3-chlorophenyl)-N′-[2-(5-methyl-1Hindol-3-yl)ethyl]-urea;
N-(3,4-dichlorophenyl)-N′-[2-(5-methyl-1Hindol-3-yl)ethyl]-urea;
N-(4-chlorophenyl)-N′-[2-(5-methyl-1Hindol-3-yl)ethyl]-urea; or
N-[2-(5-methyl-1H-indol-3-yl)ethyl]-N′-phenyl-urea.

19. The compound of claim 18 of formula

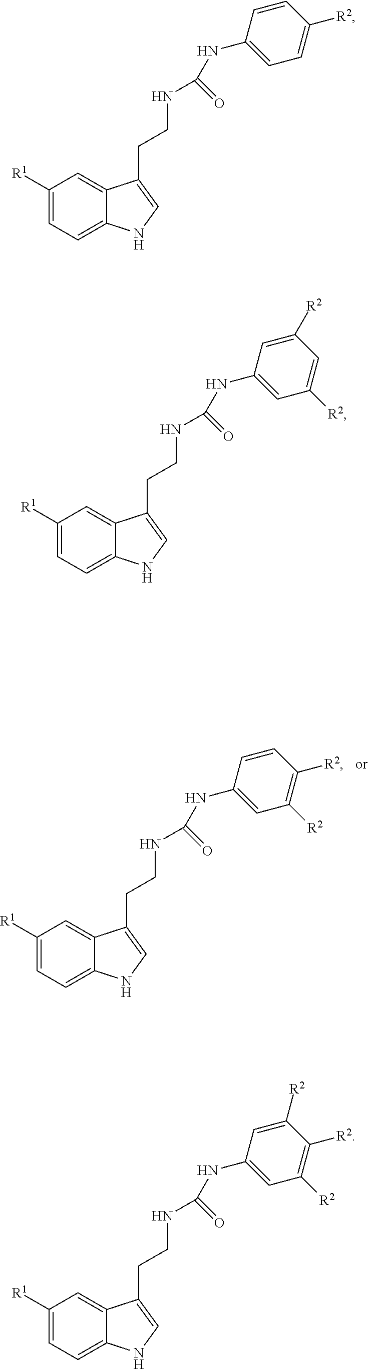

20. The compound of claim 19 of formula

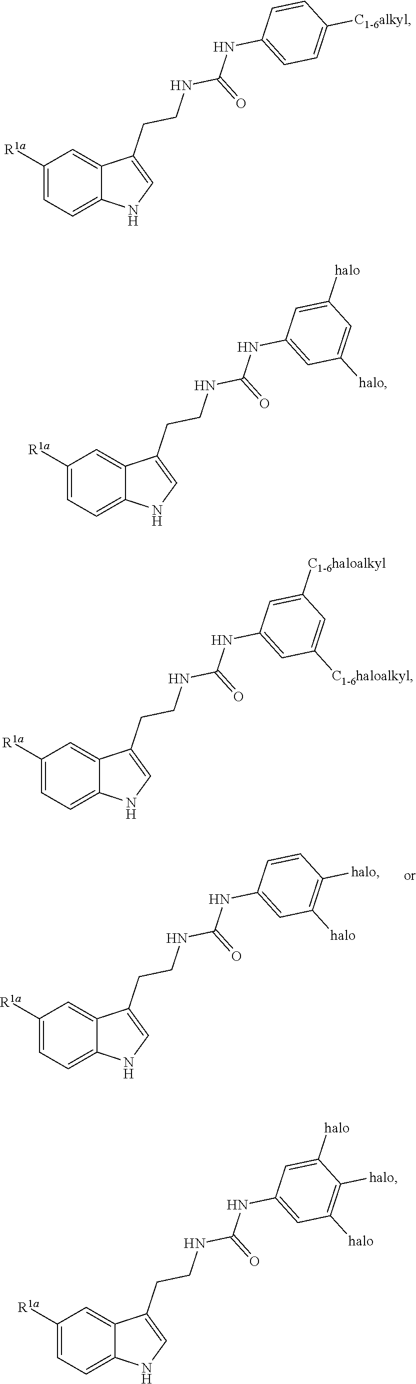

21. The compound of claim 20 of formula

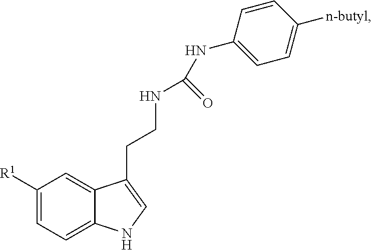

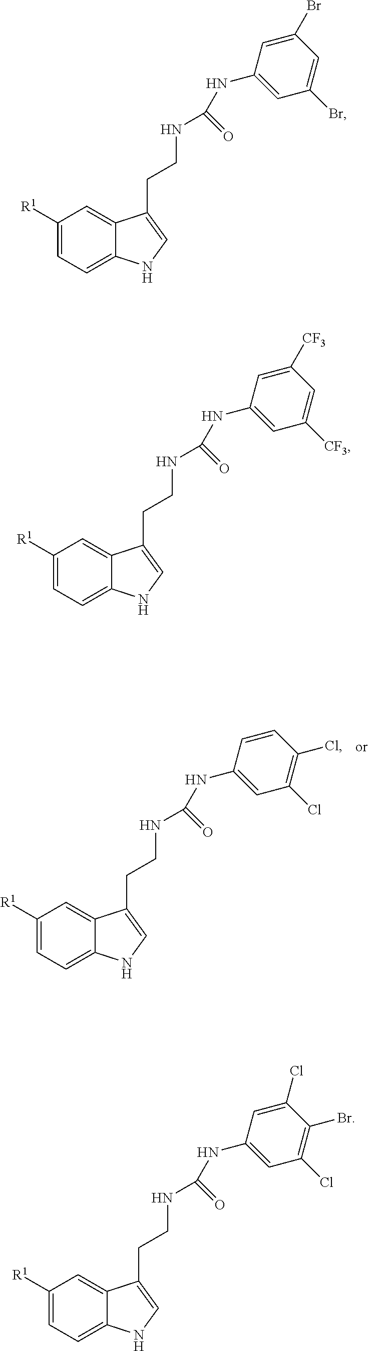

22. A compound of formula (III) 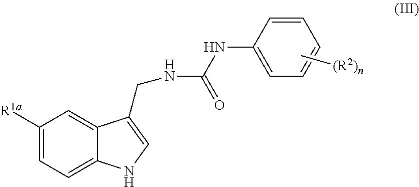
wherein
R^{1a} is hydrogen, halogen, cyano, CH₃, OCH₃, OCF₃, or OCHF₂;
R², at each occurrence, is independently halogen, cyano, C₁₋₆alkyl, C₁₋₆haloalkyl, —OC₁₋₄alkyl, —OC₁₋₄haloalkyl, —OC₁₋₃alkylene-C₃₋₆cycloalkyl, or —OC₃₋₆cycloalkyl; and
n is 0, 1, 2, 3, 4, or 5.

23. The compound of claim 22 of formula

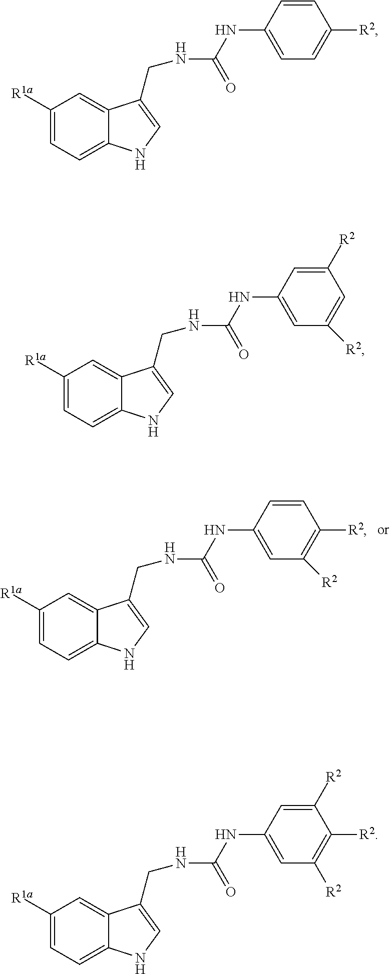

24. The compound of claim 23 of formula

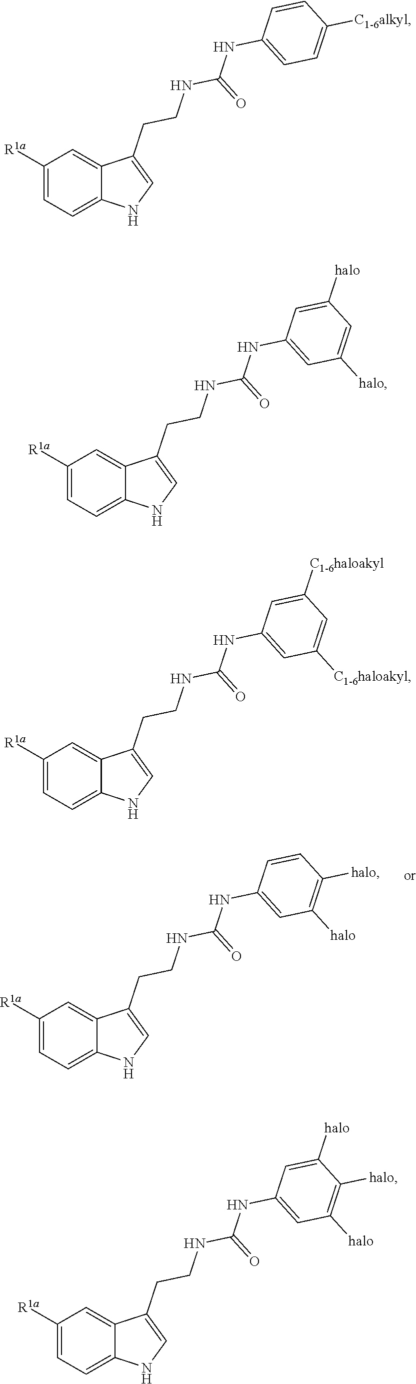

25. The compound of claim 24 of formula

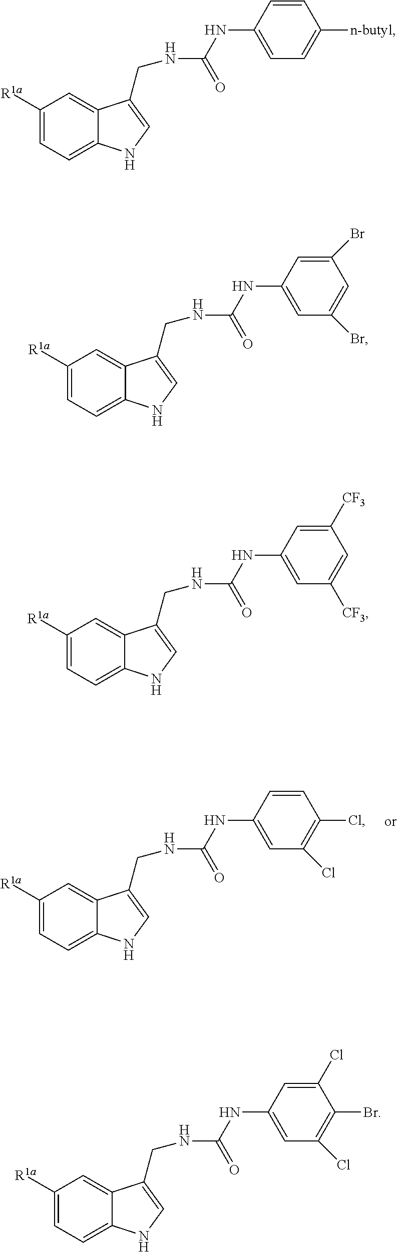

26. The compound of claim 20, wherein R¹ is halogen.

27. The compound of claim 24, wherein R^{1a} is hydrogen.

28. The compound of claim 24, wherein R^{1a} is halogen.

29. A compound selected from the group consisting of:
1-(2-(1H-indol-3-yl)ethyl)-3-(3,5-dibromophenyl)urea;
1-(2-(1H-indol-3-yl)ethyl)-3-(4-bromo-3,5-dichlorophenyl)urea;
1-(2-(5-bromo-1H-indol-3-yl)ethyl)-3-(4-butylphenyl)urea;
1-(2-(5-bromo-1H-indol-3-yl)ethyl)-3-(3,5-dibromophenyl)urea;
1-(3,5-bis(trifluoromethyl)phenyl)-3-(2-(5-bromo-1H-indol-3-yl)ethyl)urea;
1-(2-(5-bromo-1H-indol-3-yl)ethyl)-3-(3,4-dichlorophenyl)urea;
1-(2-(5-bromo-1H-indol-3-yl)ethyl)-3-(4-bromo-3,5-dichlorophenyl)urea;
1((1H-indol-3-yl)methyl)-3-(4-butylphenyl)urea;
1((1H-indol-3-yl)methyl)-3-(3,5-dibromophenyl)urea;
1((1H-indol-3-yl)methyl)-3-(3,5-bis(trifluoromethyl)phenyl)urea;
1((1H-indol-3-yl)methyl)-3-(3,4-dichlorophenyl)urea; and
1((1H-indol-3-yl)methyl)-3-(4-bromo-3,5-dichlorophenyl)urea.

30. A pharmaceutical composition comprising the compound of claim 18, and a pharmaceutically acceptable carrier.
